SEQUENCE LISTING (52 sequences)

SEQ ID NO: 1 - DNA, 771 bp, Brevibacillus laterosporus
ttatttaaattgatctgctactagttgatctaagctattctttgcatccttttcagccttagcacctgtatctttgatcgaatccttcgcttgtttctccaataatttagctgattcgtcaactactttttggatttcagcgactttttcgtcgataaagcgttttgtttcctctttagagctagcttctttctcacgaatcaaaccattaattgtctttttagcatcgttaatcgcactagttagctcttgttttgctttgtcttgcgcactggtaacattcgtttttacttcttgtcttcctagttctccagcttggttcactttattcgtaagatcgactatcgaattgacttttgtttgattggctgctttagtaatttcctgctttaatccttctaccttctggttgtattggttctgcatattgcccttgatttcattcgttttgcctctcaaagcattcaaataatcgtttttagctgccgttactgctgcaacagcacgatttgcttcttcactacctgcttgcttcacacgatcagcaacctgattctttattttattaagttcagcttcagtagctgcctttttttccgttccatacttaagcgcttcagccgtaatagacgccgatgaagcagcaaattgtttatcataccaattttttagctgcccgcctacatcaaccgccgcaaatgcagtacctaagctaccaatcagaccaactgccaacacactaatcacgattttactttttacttttttcaa

SEQ ID NO: 2 - DNA, 420 bp, Brevibacillus laterosporus
ctatttacacgggcgtacgatttcttttgttaacactttcagggcatcacgtacacttttagcgaagaccaaaggcttctcaatcttttcatagtggatataccatagattagggttggtaaatagccagtggttatgaagggcagttacctttatgccatgttctctaagtcttgaaatatacggattgatctcctcagtgagaatgaccgtctcaccaagacacaaagccctaccattcttcttactactttcaaaagaatgaaattgtgggatggttaagggagatttcgaccttctgcctaagatagtaggttttatgttggtacggagggtctgggcagtacagacaccattcacgactgtgggcttggcgttaagaatttccgcgaatctacggcaaagagggctaattttcat

SEQ ID NO: 3 - DNA, 20 bp, Artificial Sequence
  Brevibacillus laterosporus SC-CSPB complex forward primer
gcttcacacgatcagcaacc

SEQ ID NO: 4 - DNA, 20 bp, Artificial Sequence
  Brevibacillus laterosporus SC-CSPB complex reverse primer
tgtaggcgggcagctaaaaa

SEQ ID NO: 5 - DNA, 21 bp, Artificial Sequence
  Brevibacillus laterosporus SC-CSPB complex forward primer
cagcttggttcactttattcg

SEQ ID NO: 6 - DNA, 20 bp, Artificial Sequence
  Brevibacillus laterosporus SC-CSPB complex reverse primer
tgaagcaagcaggtagtgaa

SEQ ID NO: 7 - DNA, 24 bp, Artificial Sequence
  Brevibacillus laterosporus SC-CSPB complex forward primer
cgtttttacttcttgtcttcctag

SEQ ID NO: 8 - DNA, 18 bp, Artificial Sequence
  Brevibacillus laterosporus SC-CSPB complex reverse primer
aggttgctgatcgtgtga

SEQ ID NO: 9 - DNA, 22 bp, Artificial Sequence
  Brevibacillus laterosporus SC-CSPB complex reverse primer
agccttagcacctgtatctttg

SEQ ID NO: 10 - DNA, 19 bp, Artificial Sequence
  Brevibacillus laterosporus SC-CSPB complex reverse primer
aggcagctactgaagctga

SEQ ID NO: 11 - DNA, 20 bp, Artificial Sequence
  Brevibacillus laterosporus SC-CSPB complex forward primer
ctgctactagttgatctaag

SEQ ID NO: 12 - DNA, 19 bp, Artificial Sequence
  Brevibacillus laterosporus SC-CSPB complex reverse primer
ctgattggtagcttaggta

SEQ ID NO: 13 - DNA, 19 bp, Artificial Sequence
  Brevibacillus laterosporus SC-CSPB complex forward primer
ctatttacacgggcgtacg

SEQ ID NO: 14 - DNA, 24 bp, Artificial Sequence
  Brevibacillus laterosporus SC-CSPB complex reverse primer
cattcttttgaaagtagtaagaag

SEQ ID NO: 15 - DNA, 20 bp, Artificial Sequence
  Brevibacillus laterosporus SC-CSPB complex forward primer
tcaccaagacacaaagccct

SEQ ID NO: 16 - DNA, 20 bp, Artificial Sequence
  Brevibacillus laterosporus SC-CSPB complex reverse primer
ctctttgccgtagattcgcg

SEQ ID NO: 17 - DNA, 20 bp, Artificial Sequence
  Brevibacillus laterosporus SC-CSPB complex forward primer
tcagggcatcacgtacactt

SEQ ID NO: 18 - DNA, 20 bp, Artificial Sequence
  Brevibacillus laterosporus SC-CSPB complex reverse primer
gggctttgtgtcttggtgag

SEQ ID NO: 19 - protein, 256 aa, Brevibacillus laterosporus
MKKVKSKIVISVLAVGLIGSLGTAFAAVDVGGQLKNWYDKQFAASSASITAEALKYGTEKKAATEAELNKIKNQVADRVKQAGSEEANRAVAAVTAAKNDYLNALRGKTNEIKGNMQNQYNQKVEGLKQEITKAANQTKVNSIVDLTNKVNQAGELGRQEVKTNVTSAQDKAKQELTSAINDAKKTINGLIREKEASSKEETKRFIDEKVAEIQKVVDESAKLLEKQAKDSIKDTGAKAEKDAKNSLDQLVADQFK

SEQ ID NO: 20 - protein, 139 aa, Brevibacillus laterosporus
MKISPLCRRFAEILNAKPTVVNGVCTAQTLRTNIKPTILGRRSKSPLTIPQFHSFESSKKNGRALCLGETVILTEEINPYISRLREHGIKVTALHNHWLFTNPNLWYIHYEKIEKPLVFAKSVRDALKVLTKEIVRPCK

SEQ ID NO: 21 - DNA, 771 bp, Brevibacillus laterosporus
ttgaaaaaagtaaaaagtaaaatcgtgattagtgtgttggcagttggtctgattggtagcttaggtactgcatttgcggcggttgatgtaggcgggcagctaaaaaattggtatgataaacaatttgctgcttcatcggcgtctattacggctgaagcgcttaagtatggaacggaaaaaaaggcagctactgaagctgaacttaataaaataaagaatcaggttgctgatcgtgtgaagcaagcaggtagtgaagaagcaaatcgtgctgttgcagcagtaacggcagctaaaaacgattatttgaatgctttgagaggcaaaacgaatgaaatcaagggcaatatgcagaaccaatacaaccagaaggtagaaggattaaagcaggaaattactaaagcagccaatcaaacaaaagtcaattcgatagtcgatcttacgaataaagtgaaccaagctggagaactaggaagacaagaagtaaaaacgaatgttaccagtgcgcaagacaaagcaaaacaagagctaactagtgcgattaacgatgctaaaaagacaattaatggtttgattcgtgagaaagaagctagctctaaagaggaaacaaaacgctttatcgacgaaaaagtcgctgaaatccaaaaagtagttgacgaatcagctaaattattggagaaacaagcgaaggattcgatcaaagatacaggtgctaaggctgaaaaggatgcaaagaatagcttagatcaactagtagcagatcaatttaaataa

SEQ ID NO: 22 - DNA, 771 bp, Brevibacillus laterosporus
ttatttaaattgatccgctactagttgatctaagctattctttgcatccttttcagccttagcacctgtatctttgatcgaatccttcgcttgtttctccaataatttagctgattcgtcaactactttttggatttcagcgactttttcgtcgataaagcgttttgtttcctctttagagctagcttctttctcacgaatcaaaccattaattgtctttttagcatcgttaatcgcactagttagctcttgttttgctttgtcttgcgcactggtaacattcgtttttacttcttgtcttcctagttctccagcttggttcactttattcgtaagatcgactatcgaattgacttttgtttgattggctgctttagtaatttcctgctttaatccttctaccttctggttgtattggttctgcatattgcccttgatttcattcgttttgcctctcaaagcattcaaataatcgtttttagctgccgttactgctgcaacagcacgatttgcttcttcactacctgcttgcttcacacgatcagcaacctgattctttattttattaagttcagcttcagtagctgcctttttttccgttccatacttaagcgcttcagccgtaatagacgccgatgaagcagcaaattgtttatcataccaattttttagctgcccgcctacatcaaccgccgcaaatgcagtacctaagctaccaatcagaccaactgccaacacactaatcacgattttactttttacttttttcaa

SEQ ID NO: 23 - DNA, 771 bp, Brevibacillus laterosporus
ttgaaaaaagtaaaaagtaaaatcgtgattagtgtgttggcagttggtctgattggtagcttaggtactgcatttgcggcggttgatgtaggcgggcagctaaaaaattggtatgataaacaatttgctgcttcatcggcgtctattacggctgaagcgcttaagtatggaacggaaaaaaaggcagctactgaagctgaacttaataaaataaagaatcaggttgctgatcgtgtgaagcaagcaggtagtgaagaagcaaatcgtgctgttgcagcagtaacggcagctaaaaacgattatttgaatgctttgagaggcaaaacgaatgaaatcaagggcaatatgcagaaccaatacaaccagaaggtagaaggattaaagcaggaaattactaaagcagccaatcaaacaaaagtcaattcgatagtcgatcttacgaataaagtgaaccaagctggagaactaggaagacaagaagtaaaaacgaatgttaccagtgcgcaagacaaagcaaaacaagagctaactagtgcgattaacgatgctaaaaagacaattaatggtttgattcgtgagaaagaagctagctctaaagaggaaacaaaacgctttatcgacgaaaaagtcgctgaaatccaaaaagtagttgacgaatcagctaaattattggagaaacaagcgaaggattcgatcaaagatacaggtgctaaggctgaaaaggatgcaaagaatagcttagatcaactagtagcggatcaatttaaataa

SEQ ID NO: 24 - DNA, 771 bp, Brevibacillus laterosporus
ttatttaaattgatctgctactagttgatctaagctattttttgcatccttttcagccttagcacctgtatctttgatcgaatccttcgcttgtttctccaataatttagctgattcgtcaactactttttggatttcagcgactttttcgtcgataaagcgttttgtttcctctttagagctagcttctttctcacgaatcaaaccattaattgtctttttagcatcgttaatcgcactagttagctcttgttttgctttgtcttgcgcactggtaacattcgtttttacttcttgtcttcctagctctccagcttggttcactttattcgtaagatcgactatcgaattgacttttgtttgattggctgctttagtaatttcctgctttaatccttctaccttctggttgtattggttctgcatattgcccttgatttcattcgttttgcctctcaaagcattcaaataatcgtttttagctgccgttactgctgcaacagcacgatttgcttcttcactacctgcttgcttcacacgatcagcaacctgattctttattttattaagttcagcttcagtagctgcctttttttccgttccatacttaagcgcttcagccgtaatagacgccgatgaagcagcaaattgtttatcataccaattttttagctgcccgcctacatcaaccgccgcaaatgcagtacctaagctaccaatcagaccaactgccaacacactaatcacgattttactttttacttttttcaa

SEQ ID NO: 25 - DNA, 771 bp, Brevibacillus laterosporus
ttgaaaaaagtaaaaagtaaaatcgtgattagtgtgttggcagttggtctgattggtagcttaggtactgcatttgcggcggttgatgtaggcgggcagctaaaaaattggtatgataaacaatttgctgcttcatcggcgtctattacggctgaagcgcttaagtatggaacggaaaaaaaggcagctactgaagctgaacttaataaaataaagaatcaggttgctgatcgtgtgaagcaagcaggtagtgaagaagcaaatcgtgctgttgcagcagtaacggcagctaaaaacgattatttgaatgctttgagaggcaaaacgaatgaaatcaagggcaatatgcagaaccaatacaaccagaaggtagaaggattaaagcaggaaattactaaagcagccaatcaaacaaaagtcaattcgatagtcgatcttacgaataaagtgaaccaagctggagagctaggaagacaagaagtaaaaacgaatgttaccagtgcgcaagacaaagcaaaacaagagctaactagtgcgattaacgatgctaaaaagacaattaatggtttgattcgtgagaaagaagctagctctaaagaggaaacaaaacgctttatcgacgaaaaagtcgctgaaatccaaaaagtagttgacgaatcagctaaattattggagaaacaagcgaaggattcgatcaaagatacaggtgctaaggctgaaaaggatgcaaaaaatagcttagatcaactagtagcagatcaatttaaataa

SEQ ID NO: 26 - DNA, 771 bp, Brevibacillus laterosporus
ttatttaaattgatctgctactagctgatctaagctattttttgcatccttttcagccttagcacctgtatctttgatagcatccttcacttgtttctccaataatttagctgactcgtcaactactttttggatttcagctaccttttcgtcaataaagcgttttgtttcctctttcgagcttgcttccttttcacgaattaaaccattaattgtcttcttagcatcgttaatcgcactagttaattcttgttttgctttatcttgtgcactggtaacattcgttcttacttcttgtttacctagttctccagcatggttcactttattcgtaagatcaattaccgaattgacctttgtatgattagctgctttgttaatttcctgctttaatccttcgaccttctgattgtattggttctgcatattgcccttgatttcgttcgttttgcctctcaaagcattcaaataatcgtttttagcagccgttactgctgcaacagcacgatttgcttcttcactacctgcttgcttcacacggtcagcaacctgattctttattttattaagctcagcttcagtaactgcctttttttcgtttccatacttaaccgtttctgtcgtaatagacgccgctgaagcagcgaattgtttatcataccaattttttaactgcccgcctacatcaaccgccgcaaatgcagtacctaagctaccaatcagaccaactgccaacacactaatcacgattttactttttacttttttcaa

SEQ ID NO: 27 - DNA, 771 bp, Brevibacillus laterosporus
ttgaaaaaagtaaaaagtaaaatcgtgattagtgtgttggcagttggtctgattggtagcttaggtactgcatttgcggcggttgatgtaggcgggcagttaaaaaattggtatgataaacaattcgctgcttcagcggcgtctattacgacagaaacggttaagtatggaaacgaaaaaaaggcagttactgaagctgagcttaataaaataaagaatcaggttgctgaccgtgtgaagcaagcaggtagtgaagaagcaaatcgtgctgttgcagcagtaacggctgctaaaaacgattatttgaatgctttgagaggcaaaacgaacgaaatcaagggcaatatgcagaaccaatacaatcagaaggtcgaaggattaaagcaggaaattaacaaagcagctaatcatacaaaggtcaattcggtaattgatcttacgaataaagtgaaccatgctggagaactaggtaaacaagaagtaagaacgaatgttaccagtgcacaagataaagcaaaacaagaattaactagtgcgattaacgatgctaagaagacaattaatggtttaattcgtgaaaaggaagcaagctcgaaagaggaaacaaaacgctttattgacgaaaaggtagctgaaatccaaaaagtagttgacgagtcagctaaattattggagaaacaagtgaaggatgctatcaaagatacaggtgctaaggctgaaaaggatgcaaaaaatagcttagatcagctagtagcagatcaatttaaataa

SEQ ID NO: 28 - DNA, 771 bp, Brevibacillus laterosporus
ttatttaaattgatcggctactagttgatccaagctattctttgcatccttttcagccttagcacctgtatctttaatagcatccttcacttgtttctccaataatttagcggattcgtcaactactttttggatttcagcaactttttcgtcaataaagcgttttgtttcctctttagagctagcttctttctcgcgaatcaaaccattaatcgtattcttagcatcgttaatcgcactggttagttcttgctttgctttgtcttgtgcactagtaacgtttgttcttacttcttgttttcctgcttctccagcgtggttcactttatttgtaagatcgacgatcgaattgacttttgtatgattggctgctttgttaatttcctgctttaatccctctaccttctggttgtattggttctgcatattgcccttgatttcgttcgttttgcctctcaaagcattcaaataatcgtttttagcagctgttactgaagcgacggcacgatttgcttcctcgctacccgcttgcttaacacggtcagcaacctgattctttagcttattaatctcaccttctgtaattgcctttttctcgtttccgtaattaaccgcttctttcgtaatagacgatgctgaagcagcgaattgtttatcataccaatttttaagctggccacctacatcaacggccgcaaatgcagtacctaagctaccaatcagaccaactgctaacacaccaatcacgattttactttttacttttttcaa

SEQ ID NO: 29 - DNA, 771 bp, Brevibacillus laterosporus
ttgaaaaaagtaaaaagtaaaatcgtgattggtgtgttagcagttggtctgattggtagcttaggtactgcatttgcggccgttgatgtaggtggccagcttaaaaattggtatgataaacaattcgctgcttcagcatcgtctattacgaaagaagcggttaattacggaaacgagaaaaaggcaattacagaaggtgagattaataagctaaagaatcaggttgctgaccgtgttaagcaagcgggtagcgaggaagcaaatcgtgccgtcgcttcagtaacagctgctaaaaacgattatttgaatgctttgagaggcaaaacgaacgaaatcaagggcaatatgcagaaccaatacaaccagaaggtagagggattaaagcaggaaattaacaaagcagccaatcatacaaaagtcaattcgatcgtcgatcttacaaataaagtgaaccacgctggagaagcaggaaaacaagaagtaagaacaaacgttactagtgcacaagacaaagcaaagcaagaactaaccagtgcgattaacgatgctaagaatacgattaatggtttgattcgcgagaaagaagctagctctaaagaggaaacaaaacgctttattgacgaaaaagttgctgaaatccaaaaagtagttgacgaatccgctaaattattggagaaacaagtgaaggatgctattaaagatacaggtgctaaggctgaaaaggatgcaaagaatagcttggatcaactagtagccgatcaatttaaataa

SEQ ID NO: 30 - DNA, 780 bp, Brevibacillus laterosporus
ttatttaaattgatctgctactagttgatctaagctattctttgcatccttttcagccttagcacctgtatctttgatcgaatccttcgcttgtttctccaataatttagctgattcgtcaactactttttggatttcagcgactttttcgtcgataaagcgttttgtttcctctttagagctagcttctttctcacgaatcaaaccattaattgtctttttagcatcgttaatcgcactagttagctcttgttttgctttgtcttgcgcactggtaacattcgtttttacttcttgtcttcctagttctccagcttggttcactttattcgtaagatcgactatcgaattgacttttgtttgattggctgctttagtaatttcctgctttaatccttctaccttctggttgtattggttctgcatattgcccttgatttcattcgttttgcctctcaaagcattcaaataatcgtttttagctgccgttactgctgcaacagcacgatttgcttcttcactacctgcttgcttcacacgatcagcaacctgattctttattttattaagttcagcttcagtagctgcctttttttccgttccatacttaagcgcttcagccgtaatagacgccgatgaagcagcaaattgtttatcataccaattttttagctgcccgcctacatcaaccgccgcaaatgcagtacctaagctaccaatcagaccaactgccaacacactaatcacgattttactttttacttttttcaacctcttcat

SEQ ID NO: 31 - DNA, 780 bp, Brevibacillus laterosporus
atgaagaggttgaaaaaagtaaaaagtaaaatcgtgattagtgtgttggcagttggtctgattggtagcttaggtactgcatttgcggcggttgatgtaggcgggcagctaaaaaattggtatgataaacaatttgctgcttcatcggcgtctattacggctgaagcgcttaagtatggaacggaaaaaaaggcagctactgaagctgaacttaataaaataaagaatcaggttgctgatcgtgtgaagcaagcaggtagtgaagaagcaaatcgtgctgttgcagcagtaacggcagctaaaaacgattatttgaatgctttgagaggcaaaacgaatgaaatcaagggcaatatgcagaaccaatacaaccagaaggtagaaggattaaagcaggaaattactaaagcagccaatcaaacaaaagtcaattcgatagtcgatcttacgaataaagtgaaccaagctggagaactaggaagacaagaagtaaaaacgaatgttaccagtgcgcaagacaaagcaaaacaagagctaactagtgcgattaacgatgctaaaaagacaattaatggtttgattcgtgagaaagaagctagctctaaagaggaaacaaaacgctttatcgacgaaaaagtcgctgaaatccaaaaagtagttgacgaatcagctaaattattggagaaacaagcgaaggattcgatcaaagatacaggtgctaaggctgaaaaggatgcaaagaatagcttagatcaactagtagcagatcaatttaaataa

SEQ ID NO: 32 - DNA, 420 bp, Brevibacillus laterosporus
atgaaaattagccctctttgccgtagattcgcggaaattcttaacgccaagcccacagtcgtgaatggtgtctgtactgcccagaccctccgtaccaacataaaacctactatcttaggcagaaggtcgaaatctcccttaaccatcccacaatttcattcttttgaaagtagtaagaagaatggtagggctttgtgtcttggtgagacggtcattctcactgaggagatcaatccgtatatttcaagacttagagaacatggcataaaggtaactgcccttcataaccactggctatttaccaaccctaatctatggtatatccactatgaaaagattgagaagcctttggtcttcgctaaaagtgtacgtgatgccctgaaagtgttaacaaaagaaatcgtacgcccgtgtaaatag

SEQ ID NO: 33 - DNA, 420 bp, Brevibacillus laterosporus
ctatctacacgggcgtacgatttcttttgttaacactttcagggcatcacgtacacttttagcgaagaccaaaggtttctcaatcttttcatagtggatataccatagattagggttggtaaatagccagtggttatgaagggcagttacctttatgccatgttctcgaagtcttgaaatatacggattgatctcctcagtgagaatgaccgtctcaccaagacacaaagccctaccattcttcttaatgctttcaaaagaatgaaattgtgggatggttaagggagatttcgaccttctgcctaagatagtaggttttatgttggtacggagggtctgggcagtacagacaccattcacgactgtgggcttggcgttaagaatttccgcgaatctacggcaaagagggctaattttcat

SEQ ID NO: 34 - DNA, 420 bp, Brevibacillus laterosporus
atgaaaattagccctctttgccgtagattcgcggaaattcttaacgccaagcccacagtcgtgaatggtgtctgtactgcccagaccctccgtaccaacataaaacctactatcttaggcagaaggtcgaaatctcccttaaccatcccacaatttcattcttttgaaagcattaagaagaatggtagggctttgtgtcttggtgagacggtcattctcactgaggagatcaatccgtatatttcaagacttcgagaacatggcataaaggtaactgcccttcataaccactggctatttaccaaccctaatctatggtatatccactatgaaaagattgagaaacctttggtcttcgctaaaagtgtacgtgatgccctgaaagtgttaacaaaagaaatcgtacgcccgtgtagatag

SEQ ID NO: 35 - DNA, 420 bp, Brevibacillus laterosporus
ctattcacacgggcgtacgatttctttagttaacactttcagggcatcacgtacacttttagcgaagaccaaaggttcctcaagcttttcatagtggatataccatagattagggtcggtaaatagccagtggttatgaagggcagttacctttatgccatgttctcgaagtcttgaaatatacggattgatctcctcagtgagaatgactgtctcaccaagacacaaagccctacctttcttcttactgttttcaaaagaatgaaattgtgggatggttaagggagatttcgaccttctgcctaagatagtaggttttatgttggtacgaagggtctgggcagtacagacaccattaacaactgtgggctcggcgttaagaattttcgcgaatctacggcaaagagggctaattttcat

SEQ ID NO: 36 - DNA, 420 bp, Brevibacillus laterosporus
atgaaaattagccctctttgccgtagattcgcgaaaattcttaacgccgagcccacagttgttaatggtgtctgtactgcccagacccttcgtaccaacataaaacctactatcttaggcagaaggtcgaaatctcccttaaccatcccacaatttcattcttttgaaaacagtaagaagaaaggtagggctttgtgtcttggtgagacagtcattctcactgaggagatcaatccgtatatttcaagacttcgagaacatggcataaaggtaactgcccttcataaccactggctatttaccgaccctaatctatggtatatccactatgaaaagcttgaggaacctttggtcttcgctaaaagtgtacgtgatgccctgaaagtgttaactaaagaaatcgtacgcccgtgtgaatag

SEQ ID NO: 37 - DNA, 420 bp, Brevibacillus laterosporus
ctatttacacgggcgtacgatttcttttgttaacactttcagggcatcacgtacacttttagcgaagaccaaaggtttctcaatcttttcatagtggatataccatagattagggttggtaaatagccagtggttatgaagggcagttacctttatgccatgttctcgaagtcttgaaatatacggattgatctcattagtgagaatgaccgtctcaccaagacacaaagccctaccattcttcttaatgctttcaaaagaatgaaattgtgggatggttaagggagatttcgaccttctgcctaagatagtaggttttatgttggtacggagggtctgggcagtacagacaccattcacgactgtgggcttggcgttaagaatttccgcgaatctacggcaaagagggctaattttcat

SEQ ID NO: 38 - DNA, 420 bp, Brevibacillus laterosporus
atgaaaattagccctctttgccgtagattcgcggaaattcttaacgccaagcccacagtcgtgaatggtgtctgtactgcccagaccctccgtaccaacataaaacctactatcttaggcagaaggtcgaaatctcccttaaccatcccacaatttcattcttttgaaagcattaagaagaatggtagggctttgtgtcttggtgagacggtcattctcactaatgagatcaatccgtatatttcaagacttcgagaacatggcataaaggtaactgcccttcataaccactggctatttaccaaccctaatctatggtatatccactatgaaaagattgagaaacctttggtcttcgctaaaagtgtacgtgatgccctgaaagtgttaacaaaagaaatcgtacgcccgtgtaaatag

SEQ ID NO: 39 - protein, 259 aa, Brevibacillus laterosporus
MKRLKKVKSKIVISVLAVGLIGSLGTAFAAVDVGGQLKNWYDKQFAASSASITAEALKYGTEKKAATEAELNKIKNQVADRVKQAGSEEANRAVAAVTAAKNDYLNALRGKTNEIKGNMQNQYNQKVEGLKQEITKAANQTKVNSIVDLTNKVNQAGELGRQEVKTNVTSAQDKAKQELTSAINDAKKTINGLIREKEASSKEETKRFIDEKVAEIQKVVDESAKLLEKQAKDSIKDTGAKAEKDAKNSLDQLVADQFK

SEQ ID NO: 40 - protein, 256 aa, Brevibacillus laterosporus; identical to SEQ ID NO: 19

SEQ ID NO: 41 - protein, 256 aa, Brevibacillus laterosporus
LKKVKSKIVISVLAVGLIGSLGTAFAAVDVGGQLKNWYDKQFAASAASITTETVKYGNEKKAVTEAELNKIKNQVADRVKQAGSEEANRAVAAVTAAKNDYLNALRGKTNEIKGNMQNQYNQKVEGLKQEINKAANHTKVNSVIDLTNKVNHAGELGKQEVRTNVTSAQDKAKQELTSAINDAKKTINGLIREKEASSKEETKRFIDEKVAEIQKVVDESAKLLEKQVKDAIKDTGAKAEKDAKNSLDQLVADQFK

SEQ ID NO: 42 - protein, 256 aa, Brevibacillus laterosporus
LKKVKSKIVIGVLAVGLIGSLGTAFAAVDVGGQLKNWYDKQFAASASSITKEAVNYGNEKKAITEGEINKLKNQVADRVKQAGSEEANRAVASVTAAKNDYLNALRGKTNEIKGNMQNQYNQKVEGLKQEINKAANHTKVNSIVDLTNKVNHAGEAGKQEVRTNVTSAQDKAKQELTSAINDAKNTINGLIREKEASSKEETKRFIDEKVAEIQKVVDESAKLLEKQVKDAIKDTGAKAEKDAKNSLDQLVADQFK

SEQ ID NO: 43 - protein, 259 aa, Brevibacillus laterosporus; identical to SEQ ID NO: 39

SEQ ID NO: 44 - protein, 139 aa, Brevibacillus laterosporus
MKISPLCRRFAEILNAKPTVVNGVCTAQTLRTNIKPTILGRRSKSPLTIPQFHSFESIKKNGRALCLGETVILTEEINPYISRLREHGIKVTALHNHWLFTNPNLWYIHYEKIEKPLVFAKSVRDALKVLTKEIVRPCR

SEQ ID NO: 45 - protein, 139 aa, Brevibacillus laterosporus
MKISPLCRRFAKILNAEPTVVNGVCTAQTLRTNIKPTILGRRSKSPLTIPQFHSFENSKKKGRALCLGETVILTEEINPYISRLREHGIKVTALHNHWLFTDPNLWYIHYEKLEEPLVFAKSVRDALKVLTKEIVRPCE

SEQ ID NO: 46 - protein, 139 aa, Brevibacillus laterosporus
MKISPLCRRFAEILNAKPTVVNGVCTAQTLRTNIKPTILGRRSKSPLTIPQFHSFESIKKNGRALCLGETVILTNEINPYISRLREHGIKVTALHNHWLFTNPNLWYIHYEKIEKPLVFAKSVRDALKVLTKEIVRPCK

SEQ ID NO: 47 - DNA, 20 bp, Artificial Sequence
  Brevibacillus laterosporus 16S rRNA forward primer
tgtagcggtgaaatgcgtag

SEQ ID NO: 48 - DNA, 20 bp, Artificial Sequence
  Brevibacillus laterosporus 16S rRNA reverse primer
gcggcactaagggtattgaa

SEQ ID NO: 49 - DNA, 17 bp, Artificial Sequence
  Brevibacillus laterosporus 16S rRNA forward primer
tgaagcgaaacggaaag

SEQ ID NO: 50 - DNA, 20 bp, Artificial Sequence
  Brevibacillus laterosporus 16S rRNA reverse primer
cgtcaaggtgctaccttatt

SEQ ID NO: 51 - DNA, 27 bp, Artificial Sequence
  Brevibacillus laterosporus 16 S rRNA forward primer
agaccgggataacatagggaaacttat

SEQ ID NO: 52 - DNA, 25 bp, Artificial Sequence
  Brevibacillus laterosporus 16 S rRNA reverse primer
ggcatgctgatccgcgattactagc

(19) United States
(12) Patent Application Publication
(10) Pub. No.: US 2018/0044718 A1
(43) Pub. Date: Feb. 15, 2018

(54) MARKERS FOR THE DETECTION OF BREVIBACILLUS LATEROSPORUS AND RELATED METHODS AND KITS

(71) Applicant: UNIVERSITÁ DEGLI STUDI DI SASSARI, Sassari (IT)

(72) Inventors: Luca RUIU, Sassari (IT); Giovanni FLACHI, Sassari (IT); Maria Giovanna MARCHE, Sassari (IT)

(21) Appl. No.: 15/553,884

(22) PCT Filed: Feb. 25, 2016
(86) PCT No.: PCT/IT2016/000047

§ 371 Date: Aug. 25, 2017

(30) Foreign Application Priority Data
Feb. 26, 2015 (IT) RM2015A000084

(51) Int. Cl. C12Q 1/68

(52) U.S. Cl. CPC C12Q 1/689; C12Q 1/686

(57) ABSTRACT

The present invention concerns markers for the detection, in any matrix, of any Brevibacillus laterosporus strains and for the detection of specific Brevibacillus laterosporus strains.

The present invention concerns markers for the detection of Brevibacillus laterosporus and related methods and kits. Particularly, the present invention concerns markers for the detection, in any matrix, of any Brevibacillus laterosporus strains and for the detection of specific Brevibacillus laterosporus strains.

Brevibacillus laterosporus Laubach is a rod-shaped, endospore-forming bacterium morphologically characterized by the production of a typical canoe-shaped parasporal body (CSPB) firmly attached to one side of the spore, which determines its lateral position in the sporangium. It is an ubiquitous species that has been isolated from a wide range of materials including soil, gemstones, lahar, fresh water, sea water, insect bodies, leaf surfaces, locust beans, compost, milk, cheese, honey, starchy foods, gelatin-factory effluents, animal hide and wool, quails (Ruiu et al., 2013). This bacterium is considered non pathogen to humans and due to the specific properties of different strains it finds applications in diverse industrial sectors. This includes its use as a biopesticide (i.e. insecticide, fungicide, nematicide and moluscicide), a biofertilizers, in the bioremediation for pollution management, in the biomedical sectors for the production of antibiotics.

The interest on this bacterial species is raising, so that there is a need to implement methods for its detection and traceability in the environment and in the industrial production processes.

Several methods for detecting Brevibacillus laterosporus are known (EP1788093 and CN101956018) wherein oligonucleotide sequences corresponding to a 16S ribosomial RNA gene of bacteria are targeted for detection. However, 16S ribosomial RNA gene is present in a large number of bacteria with high homology level. Therefore, a detection based on this gene shows a low efficiency. In addition, the expression level of 16S ribosomial RNA gene represents a limit to the sensibility of the detection method.

In the light of above it is therefore apparent the need to provide for new methods able to overcome the disadvantages of the known methods for detecting Brevibacillus laterosporus.

Therefore, the present invention has the aim to provide a method for the detection of the bacterial species Brevibacillus laterosporus able to specifically detect the presence of B. laterosporus and to quantify it in different matrices even if the bacterial species was present in low amount.

More in detail the invention deals with a PCR-based method, preferably RT-PCR method, for the detection of the bacterium Brevibacillus laterosporus employing nucleotide sequences targeting specific genes of this species.

Target genes have been identified through a study combining a proteomic and genomic approach that highlighted the expression of these genes and the localization of proteins in the bacterial cell.

These genes encodes for proteins that can be extracted from the surface of spores contained in spore suspensions obtained through culture in different solid or liquid media (i.e. Luria Bertani Broth) with methods known in the art (Ruiu et al., 2007). Typically the surface of these spores is covered by a complex including the spore coat and the canoe shaped parasporal body (SC-CSPB-complex) that is a unique feature of this bacterial species (Fitz-James and Young, 1958). The proteins of the above mentioned complex can be extracted by alkali, but they had never been identified prior to the present invention.

Among other proteins, two proteins were identified as corresponding to proteins whose localization in the bacterial cell and function was unknown. The previous knowledge of the sequences of the genes encoding for these two proteins is the result of the whole or partial genome sequencing of strains LMG 15441, GI-9, PE36, B9, and DSM 25. The sequences and accession numbers of these genes in different strains of Brevibacillus laterosporus are shown in Examples 1 and 2. The first gene, having sequence SEQ ID NO:1 in Brevibacillus laterosporus LMG 15441, encodes for a protein with a molecular weight around 28 KDa, while the second gene, having sequence SEQ ID NO:2 in Brevibacillus laterosporus GI-9, encodes for a protein with a molecular weight around 14 kDa.

For the first time, the actual expression of these genes has been verified and the encoded proteins have been associated to the SC-CSPB complex. In addition, the inventors have discovered that alike the SC-CSPB complex, these two genes are typical of Brevibacillus laterosporus and are not found in other bacterial species.

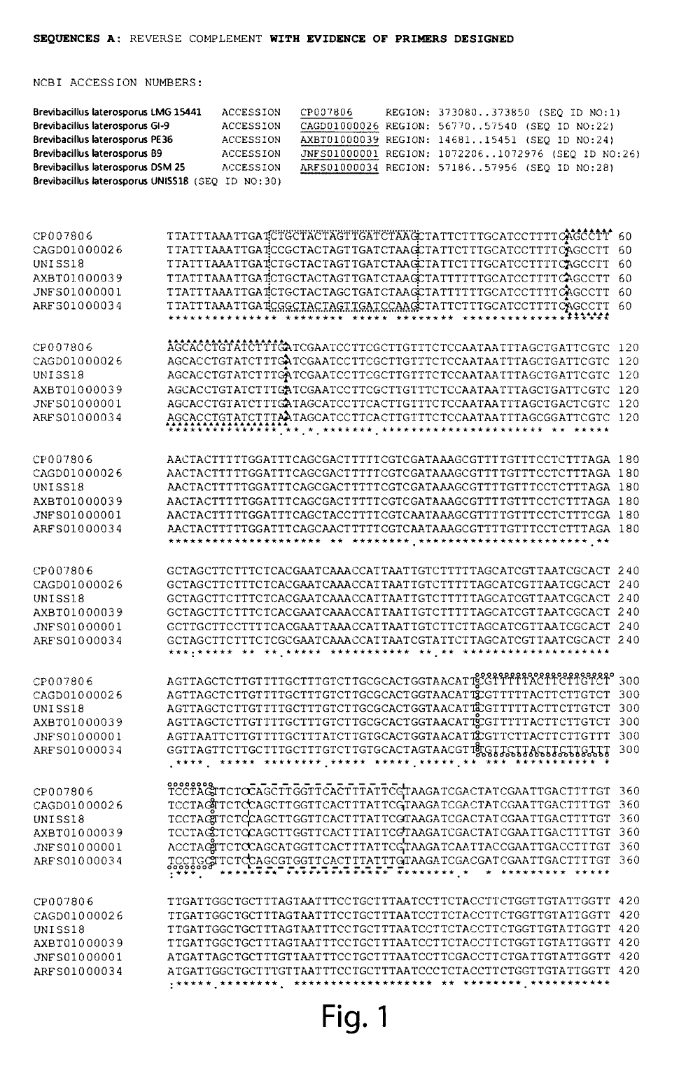

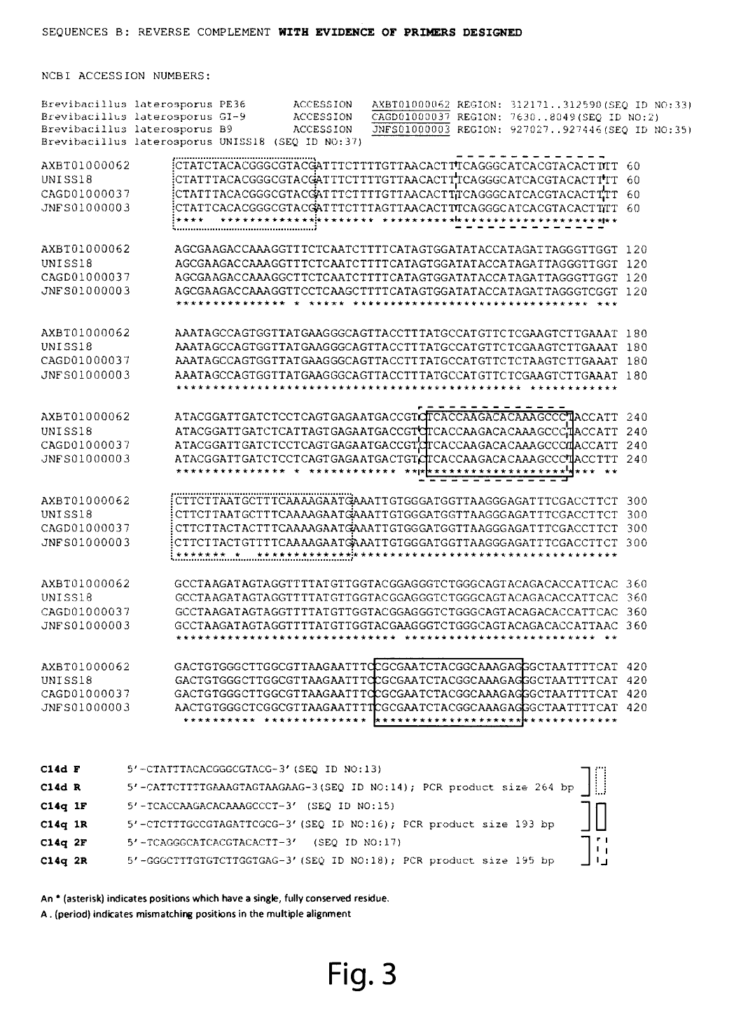

The above mentioned sequences show an high identity among the different Brevibacillus laterosporus, as shown in FIGS. 1 and 3. However, since slight differences exist among the same sequence of different strains, the nucleotide sequences encoding for a protein associated to the SC-CSPB complex and having a molecular weight around 28 KDa of different Brevibacillus laterosporus strains is hereafter reported as sequence A, whereas the nucleotide sequences encoding for a protein associated to the SC-CSPB complex and having a molecular weight around 14 KDa of different Brevibacillus laterosporus strains is hereafter reported as sequence B.

Consequently, pairs of primers designed on the nucleotide sequences of the above mentioned two genes can be used for the detection of any Brevibacillus laterosporus strains or, alternatively, for the detection of specific Brevibacillus laterosporus strains, in different matrices. More in detail, the first gene, having sequence SEQ ID NO:1 in Brevibacillus laterosporus LMG 15441 and encoding for a protein with a molecular weight around 28 KDa, can be used for the detection of any Brevibacillus laterosporus strain, while the second gene, having sequence SEQ ID NO:2 in Brevibacillus laterosporus GI-9 and encoding for a protein with a molecular weight around 14 kDa, can be used for the detection of specific Brevibacillus laterosporus strains with special reference to strain UNISS 18 deposited with NCIMB No. 41419 in the NCIMB Ltd. Aberdeen, UK and patented for the biological control of dipters (European Patent No 2,079,314; U.S. Pat. No. 8,076,119).

Any primer pairs (oligonucleotide) designed on nucleotide sequences A and B can specifically bind and amplify part or the complete genes with sequences A and B in PCR reactions if B. laterosporus genome is present as a template. As a proof of the specificity of this detection technique, if the genome of other bacterial species is used as a template, no amplification of the expected PCR product is obtained.

In addition, the detection method according to the present invention is able to detect B. laterosporus even if it was present in low amount in a sample. As showed in the examples, in fact, the inventors have found that the expression level of sequence A is high and higher than 16S ribosomial RNA gene used in the known detection methods.

The method of the invention is therefore more specific and sensitive in comparison to known methods for detecting B. laterosporus.

It is therefore specific object of the present invention a marker for use in the detection and/or quantification of Brevibacillus laterosporus in a sample, said marker consisting of a nucleic acid sequence encoding a surface polypeptide of the spore coat and the canoe shaped parasporal body of Brevibacillus laterosporus, said nucleic acid sequence comprising or consisting of:

### a) SEQ ID NO: 1;

### b) SEQ ID NO: 2;

c) a fragment of the nucleic acid sequences a) or b), said fragment having at least 12 bp, preferably from 15 to 30 bp, more preferably from 18 to 24 bp; or
d) a nucleic acid sequence having a sequence identity of at least 80%, preferably 90% with any of the nucleic acid sequences a)-c);
e) a complement nucleic sequence of said sequences a)-d)

The above mentioned identity can be determined by Basic Local Alignment search Tool (BLAST) of the National Center for Biotechnology Information (NCBI).

The nucleic acid sequences having a sequence identity of at least 80% with any of the nucleic acid sequences a)-c) are the nucleic acid sequences of different Brevibacillus laterosporus strains.

In addition, the present invention concerns a marker for use in the detection and/or quantification of Brevibacillus laterosporus in a sample, said marker consisting of a surface polypeptide sequence of the spore coat and the canoe shaped parasporal body of Brevibacillus laterosporus, said surface polypeptide sequence comprising or consisting of:

### f) SEQ ID NO: 19;

### g) SEQ ID NO: 20;

h) a fragment of the polypeptide sequence f) or g) having at least 5 aminoacids, preferably from 6 to 20 aminoacids, more preferably from 8 to 15 aminoacids;
i) a polypeptide sequence having a sequence identity of at least 90% with any of the polypeptide sequences f)-h). The above mentioned identity can be determined by Basic Local Alignment search Tool (BLAST) of the National Center for Biotechnology Information (NCBI).
The polypeptide sequences having a sequence identity of at least 90% with any of the polypeptide sequences f)-h) are the polypeptide sequences of different Brevibacillus laterosporus strains.

According to the present invention, Brevibacillus laterosporus that can be detected and/or quantified are for example Brevibacillus laterosporus ATCC9141, Brevibacillus laterosporus ATCC6456, Brevibacillus laterosporus BOD ATCC 55122, Brevibacillus laterosporus NCIMB 41419, Brevibacillus laterosporus GI-9 (Sharma et al., 2012), Brevibacillus laterosporus PE36 (Theodore et al., 2014), Brevibacillus laterosporus 89 (Li et al., 2014), Brevibacillus laterosporus DSM25 (ATCC 64).

It is further object of the present invention a method for the detection and/or quantification of Brevibacillus laterosporus in a sample, said method comprising or consisting of the detection and/or quantification of at least one marker selected from the group consisting of:

a nucleic acid sequence comprising or consisting of SEQ ID NO:1 or the complement nucleic acid sequence thereof; a fragment of said SEQ ID NO:1 or complement nucleic acid sequence thereof, said fragment having at least 12 bp, preferably from 15 to 30 bp, more preferably from 18 to 24 bp; a nucleic acid sequence having a sequence identity of at least 80%, preferably 90% with said SEQ ID NO: 1, said complement nucleic acid sequence or said fragment;

a surface polypeptide sequence comprising or consisting of SEQ ID NO: 19, a fragment thereof having at least 5 aminoacids, preferably from 6 to 20 aminoacids, more preferably from 8 to 15 aminoacids;

a polypeptide sequence having a sequence identity of at least 90% with said SEQ ID NO: 19 or fragment thereof. For example, the peptide can be detected by ELISA or Western Blot methods.

As mentioned above, the identity of the sequences can be determined by Basic Local Alignment search Tool (BLAST) of the National Center for Biotechnology Information (NCBI).

According to the present invention, Brevibacillus laterosporus that can be detected and/or quantified are for example Brevibacillus laterosporus ATCC9141, Brevibacillus laterosporus ATCC6456, Brevibacillus laterosporus BOD ATCC 55122, Brevibacillus laterosporus NCIMB 41419, Brevibacillus laterosporus GI-9 (Sharma et al., 2012), Brevibacillus laterosporus PE36 (Theodore et al., 2014), Brevibacillus laterosporus 89 (Li et al., 2014), Brevibacillus laterosporus DSM25 (ATCC 64).

A further aspect of the present invention concerns a method for the detection and/or quantification in a sample of Brevibacillus laterosporus, said method comprising or consisting of the detection of at least one marker selected from the group consisting of:

a nucleic acid sequence comprising or consisting of: SEQ ID NO:2 or the complement nucleic acid sequence thereof; a fragment of said SEQ ID NO:2 or complement nucleic acid sequence thereof, said fragment having at least 12 bp, preferably from 15 to 30 bp, more preferably from 18 to 24 bp; or

a nucleic acid sequence having a sequence identity of at least 80%, preferably 90% with said SEQ ID NO: 2, the complement nucleic acid sequence or said fragment;

a surface polypeptide sequence comprising or consisting of: SEQ ID NO: 20, a fragment thereof having at least 5 aminoacids, preferably from 6 to 20 aminoacids, more preferably from 8 to 15 aminoacids;

a polypeptide sequence having a sequence identity of at least 90% with said SEQ ID NO: 20 or fragment thereof.

For example, this method can detect Brevibacillus laterosporus NCIMB 41419.

According to a specific embodiment of the present invention, the method of claims 4-5 can be carried out by means of PCR technique or Real Time PCR by the use of at least one of the following primer pairs:

(SEQ ID NO: 3)

5′-GCTTCACACGATCAGCAACC-3′

(SEQ ID NO: 4)

5′-TGTAGGCGGGCAGCTAAAAA-3′;

(SEQ ID NO: 5)

5′-C AGC TTG GTT CAC TTT ATT CG-3′

(SEQ ID NO: 6)

5′-TG AAG CAA GCA GGT AGT GAA-3′;

(SEQ ID NO: 7)

5′-CGT TTT TAC TTC TTG TCT TCC TAG-3′

(SEQ ID NO: 8)

5′-AG GTT GCT GAT CGT GTG A-3′;

(SEQ ID NO: 9)

5′-AGC CTT AGC ACC TGT ATC TTT G-3′

(SEQ ID NO: 10)

5′-AG GCA GCT ACT GAA GCT GA-3′;

(SEQ ID NO: 11)

5′-CTGCTACTAGTTGATCTAAG-3′

(SEQ ID NO: 12)

5′-CTGATTGGTAGCTTAGGTA-3′;

preferably

(SEQ ID NO: 11)

5′-CTGCTACTAGTTGATCTAAG-3′

(SEQ ID NO: 12)

5′-CTGATTGGTAGCTTAGGTA-3′
or

(SEQ ID NO: 3)

5′-GCTTCACACGATCAGCAACC-3′

(SEQ ID NO: 4)

5′-TGTAGGCGGGCAGCTAAAAA-3′.

The primers SEQ ID NO:11 and 12 are preferable in the detection carried out by PCR, whereas primers SEQ ID NO: 3 and 4 are preferable in the detection carried out by Real Time PCR.

The method according to claims 6-7 can be carried out by means of PCR technique or by Real Time PCR by the use of at least one of the following primer pairs:

(SEQ ID NO: 13)

5′-CTA TTT ACA CGG GCG TAC G-3′

(SEQ ID NO: 14)

5′-CAT TCT TTT GAA AGTAGTAAG AAG-3′;

(SEQ ID NO: 15)

5′-TCACCAAGACACAAAGCCCT-3′

(SEQ ID NO: 16)

5′-CTCTTTGCCGTAGATTCGCG-3′;

(SEQ ID NO: 17)

5′-TCAGGGCATCACGTACACTT-3′

(SEQ ID NO: 18)

5′-GGGCTTTGTGTCTTGGTGAG-3′;

preferably

(SEQ ID NO: 13)

5′-CTA TTT ACA CGG GCG TAC G-3′

(SEQ ID NO: 14)

5′-CAT TCT TTT GAA AGTAGTAAG AAG-3′

According to a further embodiment, the present invention concerns also a method for the detection of Brevibacillus laterosporus in a sample, said method being a combination of the method as defined in anyone of the claims 4-5, 8 and the method as defined in anyone of the claims 6-7, 9.

In addition, the present invention concerns a kit for the detection and/or quantification of Brevibacillus laterosporus in a sample, said kit comprising or consisting of at least one of the following primer pairs:

(SEQ ID NO: 3)

5′-GCTTCACACGATCAGCAACC-3′

(SEQ ID NO: 4)

5′-TGTAGGCGGGCAGCTAAAAA-3′;

(SEQ ID NO: 5)

5′-C AGC TTG GTT CAC TTT ATT CG-3′

(SEQ ID NO: 6)

5′-TG AAG CAA GCA GGT AGT GAA-3′;

(SEQ ID NO: 7)

5′-CGT TTT TAC TTC TTG TCT TCC TAG-3′

(SEQ ID NO: 8)

5′-AG GTT GCT GAT CGT GTG A-3′;

(SEQ ID NO: 9)

5′-AGC CTT AGC ACC TGT ATC TTT G-3′

(SEQ ID NO: 10)

5′-AG GCA GCT ACT GAA GCT GA-3′;

(SEQ ID NO: 11)

5′-CTGCTACTAGTTGATCTAAG-3′

(SEQ ID NO: 12)

5′-CTGATTGGTAGCTTAGGTA-3′;

preferably

(SEQ ID NO: 11)

5′-CTGCTACTAGTTGATCTAAG-3′

(SEQ ID NO: 12)

5′-CTGATTGGTAGCTTAGGTA-3′-
or

(SEQ ID NO: 3)

5′-GCTTCACACGATCAGCAACC-3′

(SEQ ID NO: 4)

5′-TGTAGGCGGGCAGCTAAAAA-3′.

together with suitable reactive agents for the detection and/or quantification for instance by PCR and/or Real Time PCR.

The primers SEQ ID NO:11 and 12 are preferable in the detection carried out by PCR, whereas primers SEQ ID NO: 3 and 4 are preferable in the detection carried out by Real Time PCR.

The kit can be used for the detection and/or quantification of different Brevibacillus laterosporus strains chosen for example from the group consisting of Brevibacillus laterosporus ATCC9141, Brevibacillus laterosporus ATCC6456, Brevibacillus laterosporus BOD ATCC 55122, Brevibacillus laterosporus NCIMB 41419, Brevibacillus laterosporus GI-9 (Sharma et al., 2012), Brevibacillus laterosporus PE36 (Theodore et al., 2014), Brevibacillus laterosporus 89 (Li et al., 2014), Brevibacillus laterosporus DSM25 (ATCC 64).

In addition, the present invention concerns a kit for the detection and/or quantification in a sample of Brevibacillus laterosporus, said kit comprising or consisting of at least one of the following primer pairs:

(SEQ ID NO: 13)

5′-CTA TTT ACA CGG GCG TAC G-3′

(SEQ ID NO: 14)

5′-CAT TCT TTT GAA AGTAGTAAG AAG-3′;

(SEQ ID NO: 15)

5′-TCACCAAGACACAAAGCCCT-3′

(SEQ ID NO: 16)

5′-CTCTTTGCCGTAGATTCGCG-3′;

(SEQ ID NO: 17)

5′-TCAGGGCATCACGTACACTT-3′

(SEQ ID NO: 18)

5′-GGGCTTTGTGTCTTGGTGAG-3′;

preferably

(SEQ ID NO: 13)

5′-CTA TTT ACA CGG GCG TAC G-3′

(SEQ ID NO: 14)

5′-CAT TCT TTT GAA AGTAGTAAG AAG-3′

together with suitable reactive agents for the detection and/or quantification for example by PCR and/or Real Time PCR

Said kit can be used for detection and/or quantification for example of Brevibacillus laterosporus NCIMB 41419.

According to a further aspect of the present invention, the invention concerns a kit for the detection and/or quantification in a sample of Brevibacillus laterosporus, said kit comprising or consisting of the combination of the kit according to claims 11-12 and 13-14.

The present invention now will be described by illustrative but not limitative way according to preferred embodiment thereof with particular reference to the enclosed drawings, wherein:

FIG. 1 shows the alignment of the antisense sequences SEQ ID NO: 1, 22, 24, 26, 28 and 30 encoding for a protein associated to the SC-CSPB complex and having a molecular weight around 28 KDa in different Brevibacillus laterosporus strains.

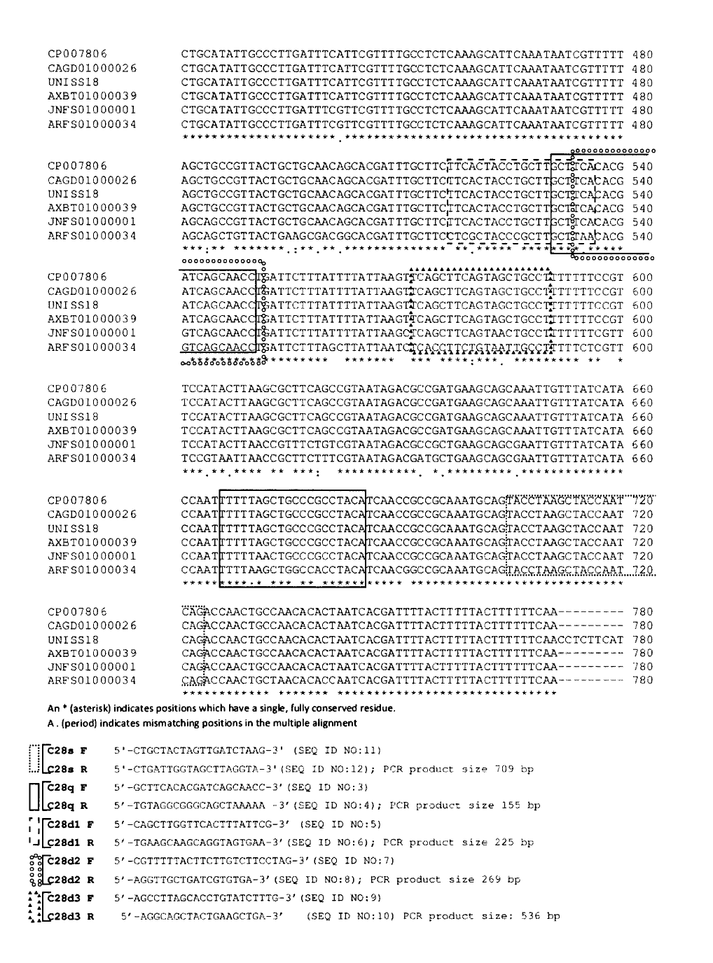

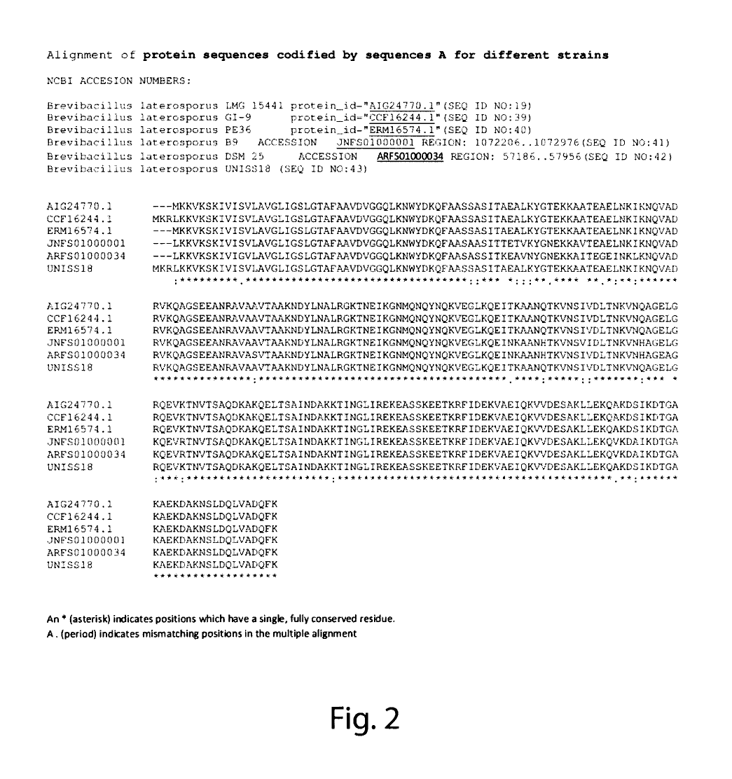

FIG. 2 shows the alignment of the polypeptide sequences SEQ ID NO: 19, 39, 40, 41, 42, 43 codified by the antisense sequences SEQ ID NO: 1, 22, 24, 26, 28 and 30 respectively.

FIG. 3 shows the alignment of the antisense sequences SEQ ID NO: 33, 2, 35, 37 encoding for a protein associated to the SC-CSPB complex and having a molecular weight around 14 kDa.

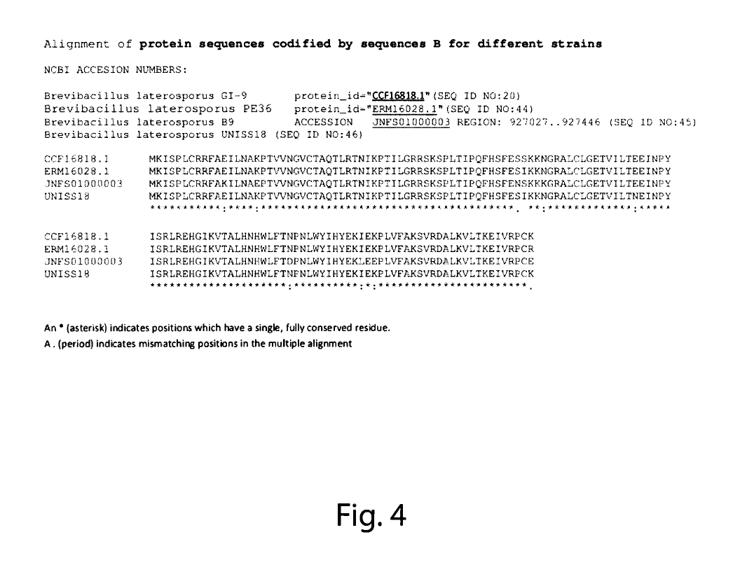

FIG. 4 shows the alignment of the polypeptide sequences SEQ ID NO: 44, 20, 45, 46 codified by the antisense sequences SEQ ID NO: 33, 2, 35, 37 respectively.

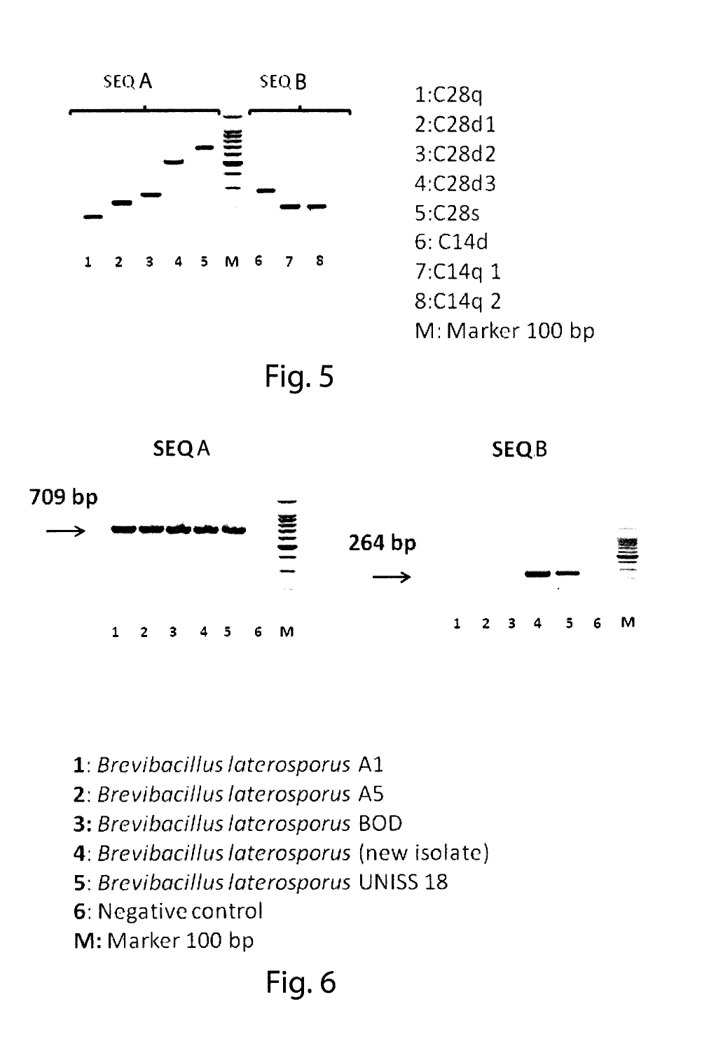

FIG. 5 shows the PCR results to detect SEQ ID NO: 30 and SEQ ID NO: 37 on Brevibacillus laterosporus UNISS 18. For SEQ ID NO: 30 the following primers pairs have been used: (1) C28q; (2) C28d1; (3) C28d2; (4) C28d3; (5) C28s; and the PCR products size obtained were: (1) 155 bp; (2) 225 bp; (3) 269 bp; (4) 536 bp; (5) 709 bp. For SEQ ID NO: 37 the following primers pair have been used: (6) C14d; (7) C14q1; (8) C14q2; and the PCR products size obtained were: (6) 264 bp; (7) 193 bp; (8) 195 bp.

FIG. 6 shows the PCR results to detect Sequences A and B on DNA extracted with commercial kit from different Brevibacillus laterosporus strains: (1) Brevibacillus laterosporus A1; (2) Brevibacillus laterosporus A5; (3) Brevibacillus laterosporus BOD; (4) Brevibacillus laterosporus (new isolate); (5) Brevibacillus laterosporus UNISS18; (6) Negative control; (M) Marker 100 bp. For Sequence A the PCR products of 709 bp were obtained using C28s primers pair; for Sequence B the PCR products of 264 bp were obtained using C14d primers pair.

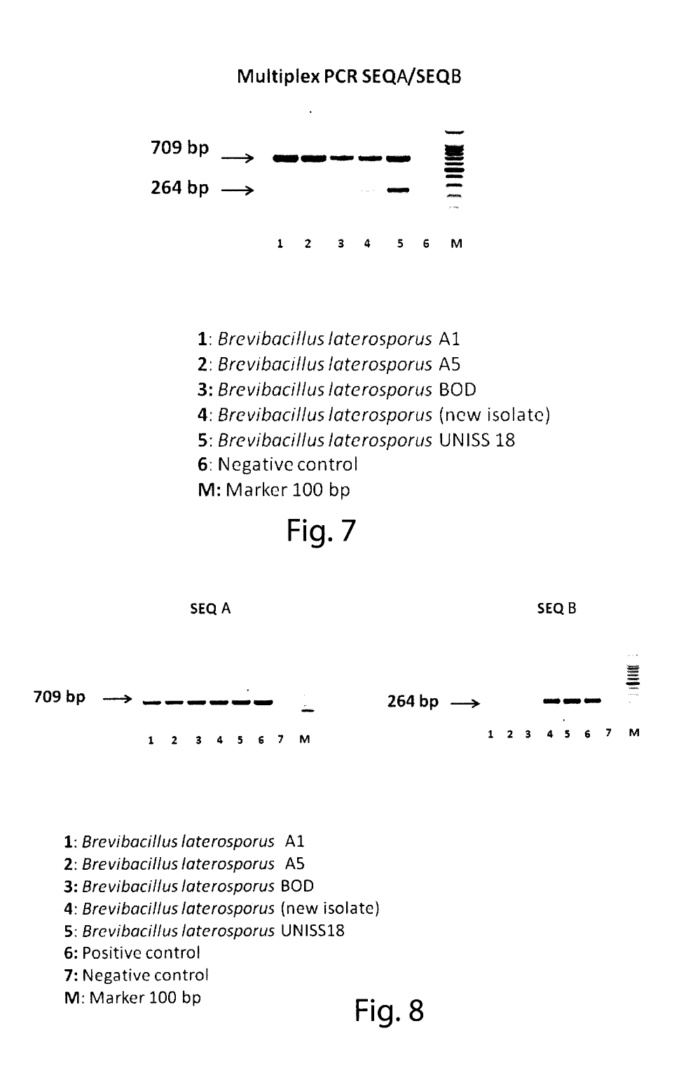

FIG. 7 shows the Multiplex PCR results to detect sequences A and B simultaneously on DNA extracted with commercial kit from different Brevibacillus laterosporus strains: (1) Brevibacillus laterosporus A1; (2) Brevibacillus laterosporus A5; (3) Brevibacillus laterosporus BOD; (4) Brevibacillus laterosporus (new isolate); (5) Brevibacillus laterosporus UNISS18; (6) Negative control; (M) Marker 100 bp. For Sequence A the PCR products of 709 bp were obtained using C28s primers pair; for Sequence B the PCR products of 264 bp were obtained using C14d primers pair.

FIG. 8 shows the PCR results to detect sequences A and B on DNA extracted with boiling method from different Brevibacillus laterosporus strains: (1) Brevibacillus laterosporus A1; (2) Brevibacillus laterosporus A5; (3) Brevibacillus laterosporus BOD; (4) Brevibacillus laterosporus (new isolate); (5) Brevibacillus laterosporus UNISS18; (6) Positive control; (7) Negative control; (M) Marker 100 bp. For sequence A the PCR products of 709 bp were obtained using C28s primers pair; for sequence B the PCR products of 264 bp were obtained using C14d primers pair.

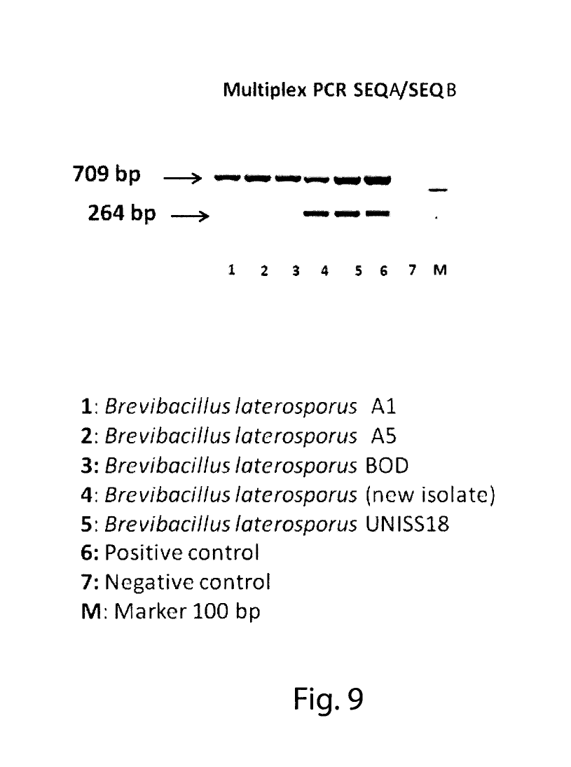

FIG. 9 shows the Multiplex PCR results to detect sequences A and B on DNA extracted with boiling method from different Brevibacillus laterosporus strains: (1) Brevibacillus laterosporus A1; (2) Brevibacillus laterosporus A5; (3) Brevibacillus laterosporus BOD; (4) Brevibacillus laterosporus (new isolate); (5) Brevibacillus laterosporus UNISS18; (6) Positive control; (7) Negative control; (M) Marker 100 bp. For sequence A the PCR products of 709 bp were obtained using C28s primers pair; for sequence B the PCR products of 264 bp were obtained using C14d primers pair.

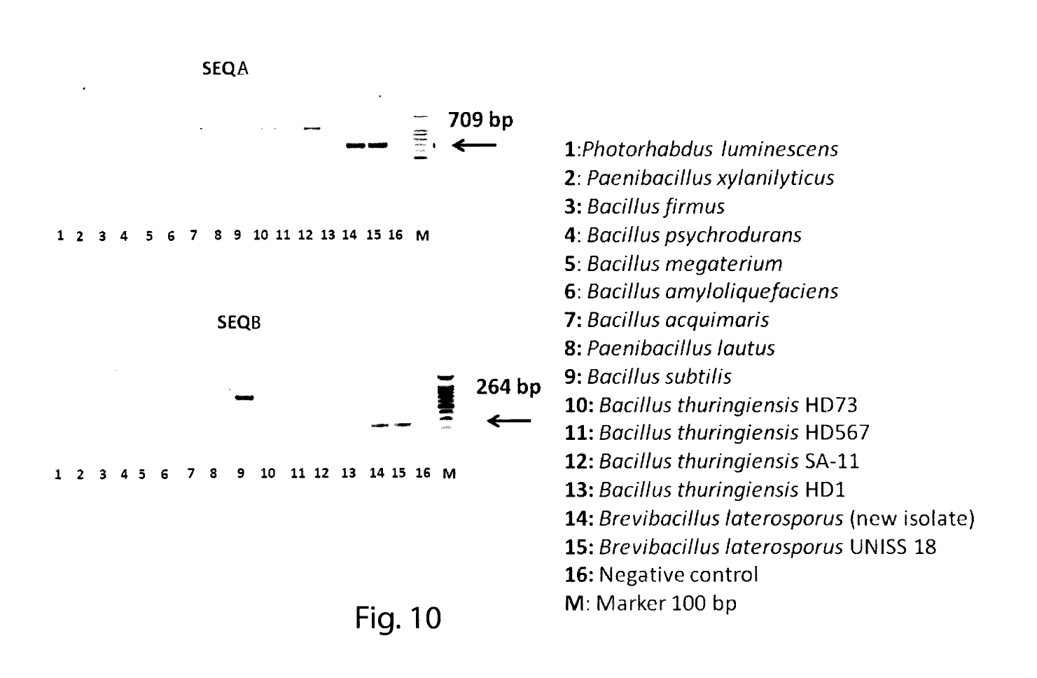

FIG. 10 shows the PCR results to detect sequences A and B on DNA extracted with commercial kit from different bacterial species: (1) Photorhabdus luminescens, (2) Paenibacillus xylanilyticus, (3) Bacillus firmus, (4) Bacillus psychrodurans, (5) Bacillus megaterium, (6) Bacillus amyloliquefaciens, (7) Bacillus acquimaris, (8) Paenibacillus lautus, (9) Bacillus subtilis, (10) Bacillus thuringiensis HD73, (11) Bacillus thuringiensis HD567, (12) Bacillus thuringiensis SA-11, (13) Bacillus thuringiensis HD1, (14) Brevibacillus laterosporus (new isolate), (15) Brevibacillus laterosporus UNISS 18 (16) Negative control; (M) Marker 100 bp. For sequence A the PCR products of 709 bp were obtained using C28s primers pair; for sequence B the PCR products of 264 bp were obtained using C14d primers pair.

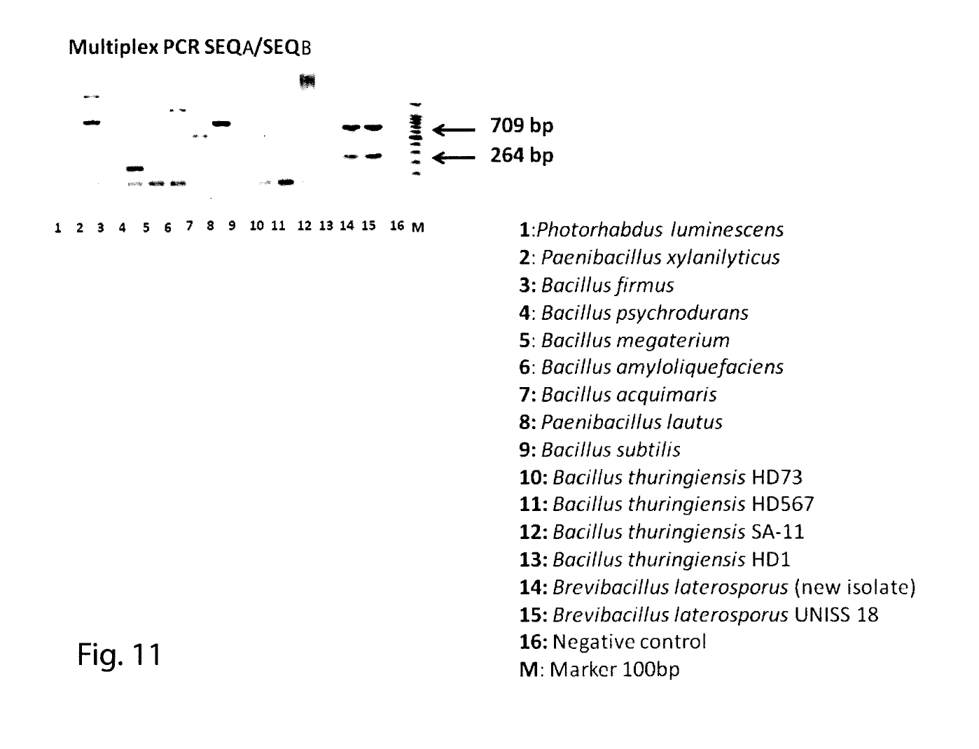

FIG. 11 shows the Multiplex PCR results to detect sequences A e B simultaneously on DNA extracted with commercial kit from different bacterial species: (1) Photorhabdus luminescens, (2) Paenibacillus xylanilyticus, (3) Bacillus firmus, (4) Bacillus psychrodurans, (5) Bacillus megaterium, (6) Bacillus amyloliquefaciens, (7) Bacillus acquimaris, (8) Paenibacillus lautus, (9) Bacillus subtilis, (10) Bacillus thuringiensis HD73, (11) Bacillus thuringiensis HD567, (12) Bacillus thuringiensis SA-11, (13) Bacillus thuringiensis HD1, (14) Brevibacillus laterosporus (new isolate), (15) Brevibacillus laterosporus UNISS 18 (16) Negative control; (M) Marker 100 bp. For sequence A the PCR products of 709 bp were obtained using C28s primers pair; for sequence B the PCR products of 264 bp were obtained using C14d primers pair.

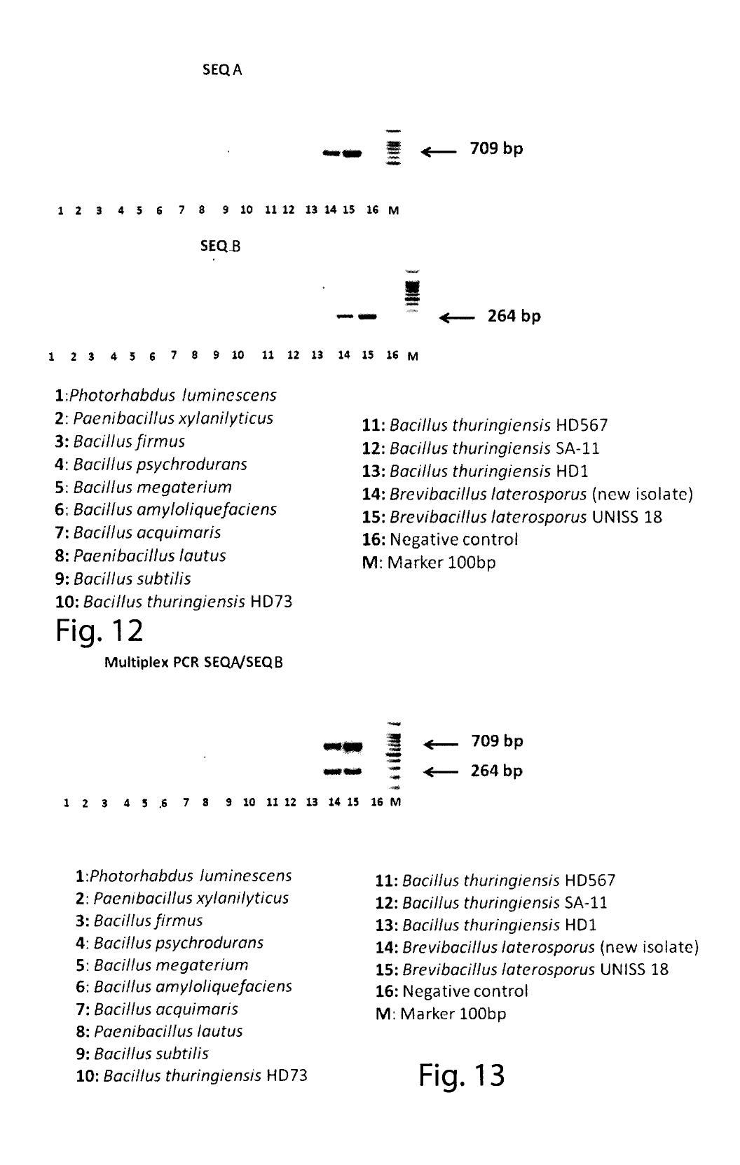

FIG. 12 shows the PCR results to detect sequences A and B on DNA extracted with boiling method from different bacterial species: (1) Photorhabdus luminescens, (2) Paenibacillus xylanilyticus, (3) Bacillus firmus, (4) Bacillus psychrodurans, (5) Bacillus megaterium, (6) Bacillus amyloliquefaciens, (7) Bacillus acquimaris, (8) Paenibacillus lautus, (9) Bacillus subtilis, (10) Bacillus thuringiensis HD73, (11) Bacillus thuringiensis HD567, (12) Bacillus thuringiensis SA-11, (13) Bacillus thuringiensis HD1, (14) Brevibacillus laterosporus (new isolate), (15) Brevibacillus laterosporus UNISS 18 (16) Negative control; (M) Marker 100 bp. For sequence A the PCR products of 709 bp were obtained using C28s primers pair; for sequence B the PCR products of 264 bp were obtained using C14d primers pair.

FIG. 13 shows the Multiplex PCR results to detect sequences A and B simultaneously on DNA extracted with boiling method from different bacterial species: (1) Photorhabdus luminescens, (2) Paenibacillus xylanilyticus, (3) Bacillus firmus, (4) Bacillus psychrodurans, (5) Bacillus megaterium, (6) Bacillus amyloliquefaciens, (7) Bacillus acquimaris, (8) Paenibacillus lautus, (9) Bacillus subtilis, (10) Bacillus thuringiensis HD73, (11) Bacillus thuringiensis HD567, (12) Bacillus thuringiensis SA-11, (13) Bacillus thuringiensis HD1, (14) Brevibacillus laterosporus (new isolate), (15) Brevibacillus laterosporus UNISS 18 (16) Negative control; (M) Marker 100 bp. For sequence A the PCR products of 709 bp were obtained using C28s primers pair; for sequence B the PCR products of 264 bp were obtained using C14d primers pair.

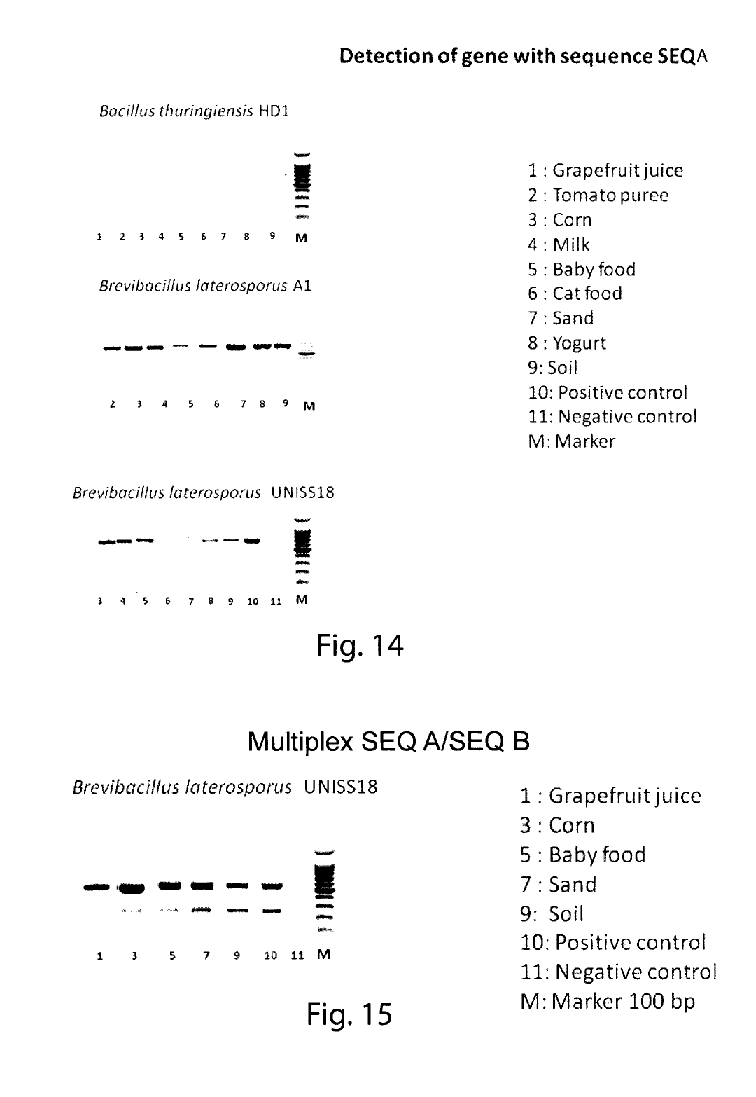

FIG. 14 shows the PCR results to detect sequence A on different matrices mixed with Bacillus thuringiensis HD1, Brevibacillus laterosporus A1 and Brevibacillus laterosporus UNISS 18. The matrices used were: (1) Grapefruit juice, (2) Tomato puree, (3) Corn, (4) Milk, (5) Baby food, (6) Cat food, (7) Sand, (8) Yougurt, (9) Soil, (10) Positive control, (11) Negative control; (M) Marker 100 bp. The sequence A PCR products of 709 bp were obtained using C28s primers pair.

FIG. 15 shows the Multiplex PCR results to detect SEQ ID NO: 30 and SEQ ID NO: 37 simultaneously on different matrices mixed with Brevibacillus laterosporus UNISS 18. The matrices used were: (1) Grapefruit juice, (3) Corn, (5) Baby food, (7) Sand, (9) Soil, (10) Positive control, (11) Negative control; (M) Marker 100 bp. For SEQ ID NO: 30 the PCR products of 709 bp were obtained using C28s primers pair; for SEQ ID NO:37 the PCR products of 264 bp were obtained using C14d primers pair.

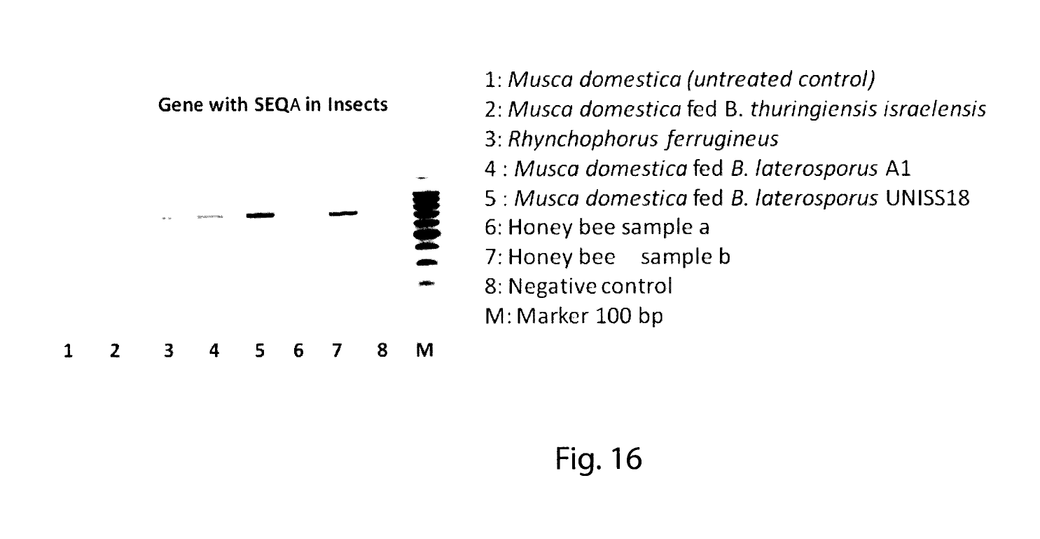

FIG. 16 shows the PCR results to detect sequence A on different insect matrices. The insects used were: (1) Musca domestica (untreated control), (2) Musca domestica fed B. thuringiensis israelensis, (3) Rhynchophorus ferrugineus, (4) Musca domestica fed Brevibacillus laterosporus A1, (5) Musca domestica fed Brevibacillus laterosporus UNISS18, (6) Honey bee sample a, (7) Honey bee sample b, (8) Negative control; (M) Marker 100 bp. Sequence A PCR products of 709 bp were obtained using C28s primers pair.

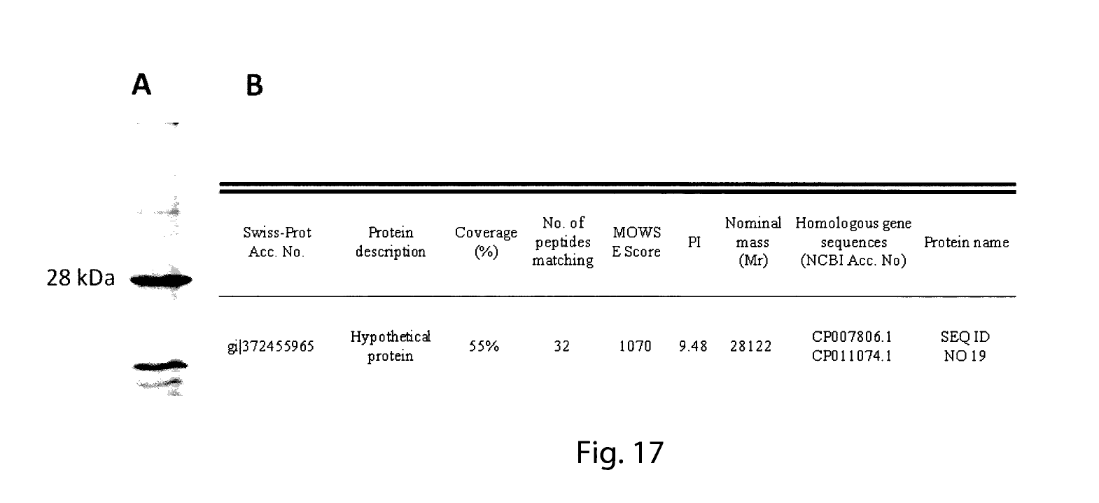

FIG. 17 shows 1-DE protein profile of Brevibacillus laterosporus SC-CSPB fraction extract showing a main band with a MW of around 28 kDa (A) and the results of its identification by LC-MS/MS (B).

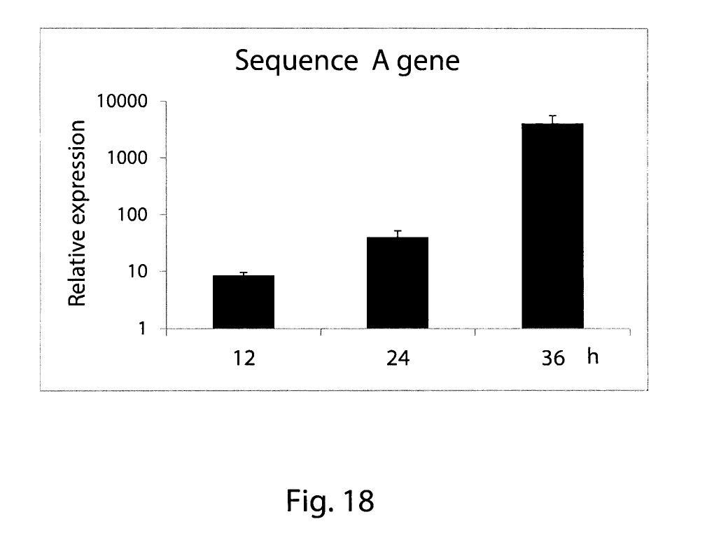

FIG. 18 shows the relative expression level of sequence A gene during different Brevibacillus laterosporus stages of growth: vegetative phase (12 h), stationary phase (24 h), sporulation phase (36 h). Relative over time changes are expressed as differences (delta) with the reference gene (16S rRNA), and are expressed as 2̂(-deltaCt).

### EXAMPLE 1: IDENTIFICATION OF TWO BREVIBACILLUS LATEROSPORUS GENES FOR ITS SPECIES-SPECIFIC DETECTION

Proteins of the complex including the spore coat and the canoe shaped parasporal body (SC-CSPB-complex) of Brevibacilus laterosporus strain NCIMB 41419 (UNISS 18) have been extracted by alkali. The proteins have been extracted by bringing the pH of a clean spore suspension to 11.5 by the careful addition of 0.2 N NaOH. Then an equal volume of 2% thioglycollic acid at the same pH was added and mixed. After 10 min incubation at room temperature, the suspension was centrifuged at 11,000 g and 4° C. for 15 min, and the supernatant was collected before being dialysed at 4° C. against 50 mM Tris-CI pH 8.0, using SnakeSkin™ Pleated Dialysis tubing, 3,500 MWCO (Cole-Parmer Instrument Company, UK). The washing buffer was changed three times after 1 h, 2 h and overnight. All insoluble material was removed from the sample by centrifugation at 20,000 g for 15 min. The final sample was analyzed by 1D-PAGE and major bands with a molecular weight ranging between 12 and 30 kDa were cut and subjected to in situ tryptic digestion before analyzes by LC MS/MS. Mass spectrometry output data were analyzed employing a Mascot server (Matrix Science, London, UK) and processed against the NCBI database (http://www.ncbi.nlm.nih.gov). In this way, among other proteins, two were identified as corresponding to hypothetical proteins whose localization in the bacterial cell and function was unknown. The previous knowledge of the sequences of the genes encoding for these two proteins is the result of the whole or partial genome sequencing of strains LMG 15441, GI-9, PE36, B9, and DSM 25.

In order to develop a system to detect Brevibacillus laterosporus among genes encoding for proteins associated to the SC-CSPB complex, two species-specific genes were identified. More specifically, one of the two sequences encodes for a protein of SC-CSPB complex with a molecular weight around 28 kDa (sequence A), while the other sequence encodes for a protein of SC-CSPB complex with a molecular weight around 14 kDa (sequence B).

Both sequences of different Brevibacillus laterosporus strains are shown below:

Sequence encoding for a protein of SC-CSPB complex with a molecular weight around 28 kDa (sequence A)

| Brevibacillus laterosporus LMG 15441 (ATCC 9141), | |
|---|---|
| GenBank: CP007806.1 | |
| ACCESSION CP007806 REGION: 373080 . . . 373850 | |
| /protein_id = “AIG24770.1” | |
| Antisense sequence | |
| SEQ ID NO: 1 | |
| TTATTTAAAT TGATCTGCTA CTAGTTGATC TAAGCTATTC TTTGCATCCT TTTCAGCCTT | 60 |
| AGCACCTGTA TCTTTGATCG AATCCTTCGC TTGTTTCTCC AATAATTTAG CTGATTCGTC | 120 |
| AACTACTTTT TGGATTTCAG CGACTTTTTC GTCGATAAAG CGTTTTGTTT CCTCTTTAGA | 180 |
| GCTAGCTTCT TTCTCACGAA TCAAACCATT AATTGTCTTT TTAGCATCGT TAATCGCACT | 240 |
| AGTTAGCTCT TGTTTTGCTT TGTCTTGCGC ACTGGTAACA TTCGTTTTTA CTTCTTGTCT | 300 |
| TCCTAGTTCT CCAGCTTGGT TCACTTTATT CGTAAGATCG ACTATCGAAT TGACTTTTGT | 360 |
| TTGATTGGCT GCTTTAGTAA TTTCCTGCTT TAATCCTTCT ACCTTCTGGT TGTATTGGTT | 420 |
| CTGCATATTG CCCTTGATTT CATTCGTTTT GCCTCTCAAA GCATTCAAAT AATCGTTTTT | 480 |
| AGCTGCCGTT ACTGCTGCAA CAGCACGATT TGCTTCTTCA CTACCTGCTT GCTTCACACG | 540 |
| ATCAGCAACC TGATTCTTTA TTTTATTAAG TTCAGCTTCA GTAGCTGCCT TTTTTTCCGT | 600 |
| TCCATACTTA AGCGCTTCAG CCGTAATAGA CGCCGATGAA GCAGCAAATT GTTTATCATA | 660 |
| CCAATTTTTT AGCTGCCCGC CTACATCAAC CGCCGCAAAT GCAGTACCTA AGCTACCAAT | 720 |
| CAGACCAACT GCCAACACAC TAATCACGAT TTTACTTTTT ACTTTTTTCA A | 771 |
| Sense sequence | |
| SEQ ID NO: 21 | |
| TTGAAAAAAG TAAAAAGTAA AATCGTGATT AGTGTGTTGG CAGTTGGTCT GATTGGTAGC | 60 |
| TTAGGTACTG CATTTGCGGC GGTTGATGTA GGCGGGCAGC TAAAAAATTG GTATGATAAA | 120 |
| CAATTTGCTG CTTCATCGGC GTCTATTACG GCTGAAGCGC TTAAGTATGG AACGGAAAAA | 180 |
| AAGGCAGCTA CTGAAGCTGA ACTTAATAAA ATAAAGAATC AGGTTGCTGA TCGTGTGAAG | 240 |
| CAAGCAGGTA GTGAAGAAGC AAATCGTGCT GTTGCAGCAG TAACGGCAGC TAAAAACGAT | 300 |
| TATTTGAATG CTTTGAGAGG CAAAACGAAT GAAATCAAGG GCAATATGCA GAACCAATAC | 360 |
| AACCAGAAGG TAGAAGGATT AAAGCAGGAA ATTACTAAAG CAGCCAATCA AACAAAAGTC | 420 |
| AATTCGATAG TCGATCTTAC GAATAAAGTG AACCAAGCTG GAGAACTAGG AAGACAAGAA | 480 |
| GTAAAAACGA ATGTTACCAG TGCGCAAGAC AAAGCAAAAC AAGAGCTAAC TAGTGCGATT | 540 |
| AACGATGCTA AAAAGACAAT TAATGGTTTG ATTCGTGAGA AAGAAGCTAG CTCTAAAGAG | 600 |
| GAAACAAAAC GCTTTATCGA CGAAAAAGTC GCTGAAATCC AAAAAGTAGT TGACGAATCA | 660 |
| GCTAAATTAT TGGAGAAACA AGCGAAGGAT TCGATCAAAG ATACAGGTGC TAAGGCTGAA | 720 |
| AAGGATGCAA AGAATAGCTT AGATCAACTA GTAGCAGATC AATTTAAATA A | 771 |
| Brevibacillus laterosporus GI-9, GenBank: GenBank: | |
| CAGD01000026.1 | |
| ACCESSION CAGD01000026 REGION: 56770 . . . 57540 | |
| /protein_id = “CCF16244.1” | |
| Antisense sequence | |
| SEQ ID NO: 22 | |
| TTATTTAAAT TGATCCGCTA CTAGTTGATC TAAGCTATTC TTTGCATCCT TTTCAGCCTT | 60 |
| AGCACCTGTA TCTTTGATCG AATCCTTCGC TTGTTTCTCC AATAATTTAG CTGATTCGTC | 120 |
| AACTACTTTT TGGATTTCAG CGACTTTTTC GTCGATAAAG CGTTTTGTTT CCTCTTTAGA | 180 |
| GCTAGCTTCT TTCTCACGAA TCAAACCATT AATTGTCTTT TTAGCATCGT TAATCGCACT | 240 |
| AGTTAGCTCT TGTTTTGCTT TGTCTTGCGC ACTGGTAACA TTCGTTTTTA CTTCTTGTCT | 300 |
| TCCTAGTTCT CCAGCTTGGT TCACTTTATT CGTAAGATCG ACTATCGAAT TGACTTTTGT | 360 |
| TTGATTGGCT GCTTTAGTAA TTTCCTGCTT TAATCCTTCT ACCTTCTGGT TGTATTGGTT | 420 |
| CTGCATATTG CCCTTGATTT CATTCGTTTT GCCTCTCAAA GCATTCAAAT AATCGTTTTT | 480 |
| AGCTGCCGTT ACTGCTGCAA CAGCACGATT TGCTTCTTCA CTACCTGCTT GCTTCACACG | 540 |
| ATCAGCAACC TGATTCTTTA TTTTATTAAG TTCAGCTTCA GTAGCTGCCT TTTTTTCCGT | 600 |
| TCCATACTTA AGCGCTTCAG CCGTAATAGA CGCCGATGAA GCAGCAAATT GTTTATCATA | 660 |
| CCAATTTTTT AGCTGCCCGC CTACATCAAC CGCCGCAAAT GCAGTACCTA AGCTACCAAT | 720 |
| CAGACCAACT GCCAACACAC TAATCACGAT TTTACTTTTT ACTTTTTTCA A | 771 |
| Sense sequence | |
| SEQ ID NO: 23 | |
| TTGAAAAAAG TAAAAAGTAA AATCGTGATT AGTGTGTTGG CAGTTGGTCT GATTGGTAGC | 60 |
| TTAGGTACTG CATTTGCGGC GGTTGATGTA GGCGGGCAGC TAAAAAATTG GTATGATAAA | 120 |
| CAATTTGCTG CTTCATCGGC GTCTATTACG GCTGAAGCGC TTAAGTATGG AACGGAAAAA | 180 |
| AAGGCAGCTA CTGAAGCTGA ACTTAATAAA ATAAAGAATC AGGTTGCTGA TCGTGTGAAG | 240 |
| CAAGCAGGTA GTGAAGAAGC AAATCGTGCT GTTGCAGCAG TAACGGCAGC TAAAAACGAT | 300 |
| TATTTGAATG CTTTGAGAGG CAAAACGAAT GAAATCAAGG GCAATATGCA GAACCAATAC | 360 |
| AACCAGAAGG TAGAAGGATT AAAGCAGGAA ATTACTAAAG CAGCCAATCA AACAAAAGTC | 420 |
| AATTCGATAG TCGATCTTAC GAATAAAGTG AACCAAGCTG GAGAACTAGG AAGACAAGAA | 480 |
| GTAAAAACGA ATGTTACCAG TGCGCAAGAC AAAGCAAAAC AAGAGCTAAC TAGTGCGATT | 540 |
| AACGATGCTA AAAAGACAAT TAATGGTTTG ATTCGTGAGA AAGAAGCTAG CTCTAAAGAG | 600 |
| GAAACAAAAC GCTTTATCGA CGAAAAAGTC GCTGAAATCC AAAAAGTAGT TGACGAATCA | 660 |
| GCTAAATTAT TGGAGAAACA AGCGAAGGAT TCGATCAAAG ATACAGGTGC TAAGGCTGAA | 720 |
| AAGGATGCAA AGAATAGCTT AGATCAACTA GTAGCGGATC AATTTAAATA A | 771 |
| Brevibacillus laterosporus PE36, GenBank: AXBT01000039.1 | |
| ACCESSION AXBT01000039 REGION: 14681 . . . 15451 | |
| /protein_id = “ERM16574.1” | |
| Antisense sequence | |
| SEQ ID NO: 24 | |
| TTATTTAAAT TGATCTGCTA CTAGTTGATC TAAGCTATTT TTTGCATCCT TTTCAGCCTT | 60 |
| AGCACCTGTA TCTTTGATCG AATCCTTCGC TTGTTTCTCC AATAATTTAG CTGATTCGTC | 120 |
| AACTACTTTT TGGATTTCAG CGACTTTTTC GTCGATAAAG CGTTTTGTTT CCTCTTTAGA | 180 |
| GCTAGCTTCT TTCTCACGAA TCAAACCATT AATTGTCTTT TTAGCATCGT TAATCGCACT | 240 |
| AGTTAGCTCT TGTTTTGCTT TGTCTTGCGC ACTGGTAACA TTCGTTTTTA CTTCTTGTCT | 300 |
| TCCTAGCTCT CCAGCTTGGT TCACTTTATT CGTAAGATCG ACTATCGAAT TGACTTTTGT | 360 |
| TTGATTGGCT GCTTTAGTAA TTTCCTGCTT TAATCCTTCT ACCTTCTGGT TGTATTGGTT | 420 |
| CTGCATATTG CCCTTGATTT CATTCGTTTT GCCTCTCAAA GCATTCAAAT AATCGTTTTT | 480 |
| AGCTGCCGTT ACTGCTGCAA CAGCACGATT TGCTTCTTCA CTACCTGCTT GCTTCACACG | 540 |
| ATCAGCAACC TGATTCTTTA TTTTATTAAG TTCAGCTTCA GTAGCTGCCT TTTTTTCCGT | 600 |
| TCCATACTTA AGCGCTTCAG CCGTAATAGA CGCCGATGAA GCAGCAAATT GTTTATCATA | 660 |
| CCAATTTTTT AGCTGCCCGC CTACATCAAC CGCCGCAAAT GCAGTACCTA AGCTACCAAT | 720 |
| CAGACCAACT GCCAACACAC TAATCACGAT TTTACTTTTT ACTTTTTTCA A | 771 |
| Sense sequence | |
| SEQ ID NO: 25 | |
| TTGAAAAAAG TAAAAAGTAA AATCGTGATT AGTGTGTTGG CAGTTGGTCT GATTGGTAGC | 60 |
| TTAGGTACTG CATTTGCGGC GGTTGATGTA GGCGGGCAGC TAAAAAATTG GTATGATAAA | 120 |
| CAATTTGCTG CTTCATCGGC GTCTATTACG GCTGAAGCGC TTAAGTATGG AACGGAAAAA | 180 |
| AAGGCAGCTA CTGAAGCTGA ACTTAATAAA ATAAAGAATC AGGTTGCTGA TCGTGTGAAG | 240 |
| CAAGCAGGTA GTGAAGAAGC AAATCGTGCT GTTGCAGCAG TAACGGCAGC TAAAAACGAT | 300 |
| TATTTGAATG CTTTGAGAGG CAAAACGAAT GAAATCAAGG GCAATATGCA GAACCAATAC | 360 |
| AACCAGAAGG TAGAAGGATT AAAGCAGGAA ATTACTAAAG CAGCCAATCA AACAAAAGTC | 420 |
| AATTCGATAG TCGATCTTAC GAATAAAGTG AACCAAGCTG GAGAGCTAGG AAGACAAGAA | 480 |
| GTAAAAACGA ATGTTACCAG TGCGCAAGAC AAAGCAAAAC AAGAGCTAAC TAGTGCGATT | 540 |
| AACGATGCTA AAAAGACAAT TAATGGTTTG ATTCGTGAGA AAGAAGCTAG CTCTAAAGAG | 600 |
| GAAACAAAAC GCTTTATCGA CGAAAAAGTC GCTGAAATCC AAAAAGTAGT TGACGAATCA | 660 |
| GCTAAATTAT TGGAGAAACA AGCGAAGGAT TCGATCAAAG ATACAGGTGC TAAGGCTGAA | 720 |
| AAGGATGCAA AAAATAGCTT AGATCAACTA GTAGCAGATC AATTTAAATA A | 771 |
| Brevibacillus laterosporus strain B9, GenBank: | |
| JNFS01000001.1 | |
| ACCESSION JNFS01000001 REGION: 1072206 . . . 1072976 | |
| Antisense sequence | |
| SEQ ID NO: 26 | |
| TTATTTAAAT TGATCTGCTA CTAGCTGATC TAAGCTATTT TTTGCATCCT TTTCAGCCTT | 60 |
| AGCACCTGTA TCTTTGATAG CATCCTTCAC TTGTTTCTCC AATAATTTAG CTGACTCGTC | 120 |
| AACTACTTTT TGGATTTCAG CTACCTTTTC GTCAATAAAG CGTTTTGTTT CCTCTTTCGA | 180 |
| GCTTGCTTCC TTTTCACGAA TTAAACCATT AATTGTCTTC TTAGCATCGT TAATCGCACT | 240 |
| AGTTAATTCT TGTTTTGCTT TATCTTGTGC ACTGGTAACA TTCGTTCTTA CTTCTTGTTT | 300 |
| ACCTAGTTCT CCAGCATGGT TCACTTTATT CGTAAGATCA ATTACCGAAT TGACCTTTGT | 360 |
| ATGATTAGCT GCTTTGTTAA TTTCCTGCTT TAATCCTTCG ACCTTCTGAT TGTATTGGTT | 420 |
| CTGCATATTG CCCTTGATTT CGTTCGTTTT GCCTCTCAAA GCATTCAAAT AATCGTTTTT | 480 |
| AGCAGCCGTT ACTGCTGCAA CAGCACGATT TGCTTCTTCA CTACCTGCTT GCTTCACACG | 540 |
| GTCAGCAACC TGATTCTTTA TTTTATTAAG CTCAGCTTCA GTAACTGCCT TTTTTTCGTT | 600 |
| TCCATACTTA ACCGTTTCTG TCGTAATAGA CGCCGCTGAA GCAGCGAATT GTTTATCATA | 660 |
| CCAATTTTTT AACTGCCCGC CTACATCAAC CGCCGCAAAT GCAGTACCTA AGCTACCAAT | 720 |
| CAGACCAACT GCCAACACAC TAATCACGAT TTTACTTTTT ACTTTTTTCA A | 771 |
| Sense sequence | |
| SEQ ID NO: 27 | |
| TTGAAAAAAG TAAAAAGTAA AATCGTGATT AGTGTGTTGG CAGTTGGTCT GATTGGTAGC | 60 |
| TTAGGTACTG CATTTGCGGC GGTTGATGTA GGCGGGCAGT TAAAAAATTG GTATGATAAA | 120 |
| CAATTCGCTG CTTCAGCGGC GTCTATTACG ACAGAAACGG TTAAGTATGG AAACGAAAAA | 180 |
| AAGGCAGTTA CTGAAGCTGA GCTTAATAAA ATAAAGAATC AGGTTGCTGA CCGTGTGAAG | 240 |
| CAAGCAGGTA GTGAAGAAGC AAATCGTGCT GTTGCAGCAG TAACGGCTGC TAAAAACGAT | 300 |
| TATTTGAATG CTTTGAGAGG CAAAACGAAC GAAATCAAGG GCAATATGCA GAACCAATAC | 360 |
| AATCAGAAGG TCGAAGGATT AAAGCAGGAA ATTAACAAAG CAGCTAATCA TACAAAGGTC | 420 |
| AATTCGGTAA TTGATCTTAC GAATAAAGTG AACCATGCTG GAGAACTAGG TAAACAAGAA | 480 |
| GTAAGAACGA ATGTTACCAG TGCACAAGAT AAAGCAAAAC AAGAATTAAC TAGTGCGATT | 540 |
| AACGATGCTA AGAAGACAAT TAATGGTTTA ATTCGTGAAA AGGAAGCAAG CTCGAAAGAG | 600 |
| GAAACAAAAC GCTTTATTGA CGAAAAGGTA GCTGAAATCC AAAAAGTAGT TGACGAGTCA | 660 |
| GCTAAATTAT TGGAGAAACA AGTGAAGGAT GCTATCAAAG ATACAGGTGC TAAGGCTGAA | 720 |
| AAGGATGCAA AAAATAGCTT AGATCAGCTA GTAGCAGATC AATTTAAATA A | 771 |
| Brevibacillus laterosporus DSM 25, GenBank: | |
| ARFS01000034.1 | |
| ACCESSION ARFS01000034 REGION: 57186 . . . 57956 | |
| Antisense sequence | |
| SEQ ID NO: 28 | |
| TTATTTAAAT TGATCGGCTA CTAGTTGATC CAAGCTATTC TTTGCATCCT TTTCAGCCTT | 60 |
| AGCACCTGTA TCTTTAATAG CATCCTTCAC TTGTTTCTCC AATAATTTAG CGGATTCGTC | 120 |
| AACTACTTTT TGGATTTCAG CAACTTTTTC GTCAATAAAG CGTTTTGTTT CCTCTTTAGA | 180 |
| GCTAGCTTCT TTCTCGCGAA TCAAACCATT AATCGTATTC TTAGCATCGT TAATCGCACT | 240 |
| GGTTAGTTCT TGCTTTGCTT TGTCTTGTGC ACTAGTAACG TTTGTTCTTA CTTCTTGTTT | 300 |
| TCCTGCTTCT CCAGCGTGGT TCACTTTATT TGTAAGATCG ACGATCGAAT TGACTTTTGT | 360 |
| ATGATTGGCT GCTTTGTTAA TTTCCTGCTT TAATCCCTCT ACCTTCTGGT TGTATTGGTT | 420 |
| CTGCATATTG CCCTTGATTT CGTTCGTTTT GCCTCTCAAA GCATTCAAAT AATCGTTTTT | 480 |
| AGCAGCTGTT ACTGAAGCGA CGGCACGATT TGCTTCCTCG CTACCCGCTT GCTTAACACG | 540 |
| GTCAGCAACC TGATTCTTTA GCTTATTAAT CTCACCTTCT GTAATTGCCT TTTTCTCGTT | 600 |
| TCCGTAATTA ACCGCTTCTT TCGTAATAGA CGATGCTGAA GCAGCGAATT GTTTATCATA | 660 |
| CCAATTTTTA AGCTGGCCAC CTACATCAAC GGCCGCAAAT GCAGTACCTA AGCTACCAAT | 720 |
| CAGACCAACT GCTAACACAC CAATCACGAT TTTACTTTTT ACTTTTTTCA A | 771 |
| Sense sequence | |
| SEQ ID NO: 29 | |
| TTGAAAAAAG TAAAAAGTAA AATCGTGATT GGTGTGTTAG CAGTTGGTCT GATTGGTAGC | 60 |
| TTAGGTACTG CATTTGCGGC CGTTGATGTA GGTGGCCAGC TTAAAAATTG GTATGATAAA | 120 |
| CAATTCGCTG CTTCAGCATC GTCTATTACG AAAGAAGCGG TTAATTACGG AAACGAGAAA | 180 |
| AAGGCAATTA CAGAAGGTGA GATTAATAAG CTAAAGAATC AGGTTGCTGA CCGTGTTAAG | 240 |
| CAAGCGGGTA GCGAGGAAGC AAATCGTGCC GTCGCTTCAG TAACAGCTGC TAAAAACGAT | 300 |
| TATTTGAATG CTTTGAGAGG CAAAACGAAC GAAATCAAGG GCAATATGCA GAACCAATAC | 360 |
| AACCAGAAGG TAGAGGGATT AAAGCAGGAA ATTAACAAAG CAGCCAATCA TACAAAAGTC | 420 |
| AATTCGATCG TCGATCTTAC AAATAAAGTG AACCACGCTG GAGAAGCAGG AAAACAAGAA | 480 |
| GTAAGAACAA ACGTTACTAG TGCACAAGAC AAAGCAAAGC AAGAACTAAC CAGTGCGATT | 540 |
| AACGATGCTA AGAATACGAT TAATGGTTTG ATTCGCGAGA AAGAAGCTAG CTCTAAAGAG | 600 |
| GAAACAAAAC GCTTTATTGA CGAAAAAGTT GCTGAAATCC AAAAAGTAGT TGACGAATCC | 660 |
| GCTAAATTAT TGGAGAAACA AGTGAAGGAT GCTATTAAAG ATACAGGTGC TAAGGCTGAA | 720 |
| AAGGATGCAA AGAATAGCTT GGATCAACTA GTAGCCGATC AATTTAAATA A | 771 |
| Brevibacillus laterosporus NCIMB 41419 (UNISS 18) | |
| Antisense sequence | |
| (SEQ ID NO: 30) | |
| TTATTTAAAT TGATCTGCTA CTAGTTGATC TAAGCTATTC TTTGCATCCT TTTCAGCCTT | 60 |
| AGCACCTGTA TCTTTGATCG AATCCTTCGC TTGTTTCTCC AATAATTTAG CTGATTCGTC | 120 |
| AACTACTTTT TGGATTTCAG CGACTTTTTC GTCGATAAAG CGTTTTGTTT CCTCTTTAGA | 180 |
| GCTAGCTTCT TTCTCACGAA TCAAACCATT AATTGTCTTT TTAGCATCGT TAATCGCACT | 240 |
| AGTTAGCTCT TGTTTTGCTT TGTCTTGCGC ACTGGTAACA TTCGTTTTTA CTTCTTGTCT | 300 |
| TCCTAGTTCT CCAGCTTGGT TCACTTTATT CGTAAGATCG ACTATCGAAT TGACTTTTGT | 360 |
| TTGATTGGCT GCTTTAGTAA TTTCCTGCTT TAATCCTTCT ACCTTCTGGT TGTATTGGTT | 420 |
| CTGCATATTG CCCTTGATTT CATTCGTTTT GCCTCTCAAA GCATTCAAAT AATCGTTTTT | 480 |
| AGCTGCCGTT ACTGCTGCAA CAGCACGATT TGCTTCTTCA CTACCTGCTT GCTTCACACG | 540 |
| ATCAGCAACC TGATTCTTTA TTTTATTAAG TTCAGCTTCA GTAGCTGCCT TTTTTTCCGT | 600 |
| TCCATACTTA AGCGCTTCAG CCGTAATAGA CGCCGATGAA GCAGCAAATT GTTTATCATA | 660 |
| CCAATTTTTT AGCTGCCCGC CTACATCAAC CGCCGCAAAT GCAGTACCTA AGCTACCAAT | 720 |
| CAGACCAACT GCCAACACAC TAATCACGAT TTTACTTTTT ACTTTTTTCA ACCTCTTCAT | 780 |
| UNISS 18 Sense sequence | |
| SEQ ID NO: 31 | |
| ATGAAGAGGT TGAAAAAAGT AAAAAGTAAA ATCGTGATTA GTGTGTTGGC AGTTGGTCTG | 60 |
| ATTGGTAGCT TAGGTACTGC ATTTGCGGCG GTTGATGTAG GCGGGCAGCT AAAAAATTGG | 120 |
| TATGATAAAC AATTTGCTGC TTCATCGGCG TCTATTACGG CTGAAGCGCT TAAGTATGGA | 180 |
| ACGGAAAAAA AGGCAGCTAC TGAAGCTGAA CTTAATAAAA TAAAGAATCA GGTTGCTGAT | 240 |
| CGTGTGAAGC AAGCAGGTAG TGAAGAAGCA AATCGTGCTG TTGCAGCAGT AACGGCAGCT | 300 |
| AAAAACGATT ATTTGAATGC TTTGAGAGGC AAAACGAATG AAATCAAGGG CAATATGCAG | 360 |
| AACCAATACA ACCAGAAGGT AGAAGGATTA AAGCAGGAAA TTACTAAAGC AGCCAATCAA | 420 |
| ACAAAAGTCA ATTCGATAGT CGATCTTACG AATAAAGTGA ACCAAGCTGG AGAACTAGGA | 480 |
| AGACAAGAAG TAAAAACGAA TGTTACCAGT GCGCAAGACA AAGCAAAACA AGAGCTAACT | 540 |
| AGTGCGATTA ACGATGCTAA AAAGACAATT AATGGTTTGA TTCGTGAGAA AGAAGCTAGC | 600 |
| TCTAAAGAGG AAACAAAACG CTTTATCGAC GAAAAAGTCG CTGAAATCCA AAAAGTAGTT | 660 |
| GACGAATCAG CTAAATTATT GGAGAAACAA GCGAAGGATT CGATCAAAGA TACAGGTGCT | 720 |
| AAGGCTGAAA AGGATGCAAA GAATAGCTTA GATCAACTAG TAGCAGATCA ATTTAAATAA | 780 |

FIG. 1 shows the alignment of the above-mentioned antisense sequences proving that the sequences have high identity among different Brevibacillus laterosporus strains. The alignment has been carried out by Basic Local Alignment-Search Tool (BLAST) of the National Center for Biotechnology Information (NCBI).

In addition, FIG. 2 shows the alignment of the polypeptide sequences codified by the above mentioned nucleic sequences.

Sequence Encoding for a Protein of SC-CSPB Complex with a Molecular Weight Around 14 kDa (Sequence B)

| Brevibacillus laterosporus GI-9, GenBank: CAGD01000037.1 | |
|---|---|
| ACCESSION CAGD01000037 REGION: 7630 . . . 8049 | |
| /protein_id = “CCF16818.1” | |
| Antisense sequence | |
| SEQ ID NO: 2 | |
| CTATTTACAC GGGCGTACGA TTTCTTTTGT TAACACTTTC AGGGCATCAC GTACACTTTT | 60 |
| AGCGAAGACC AAAGGCTTCT CAATCTTTTC ATAGTGGATA TACCATAGAT TAGGGTTGGT | 120 |
| AAATAGCCAG TGGTTATGAA GGGCAGTTAC CTTTATGCCA TGTTCTCTAA GTCTTGAAAT | 180 |
| ATACGGATTG ATCTCCTCAG TGAGAATGAC CGTCTCACCA AGACACAAAG CCCTACCATT | 240 |
| CTTCTTACTA CTTTCAAAAG AATGAAATTG TGGGATGGTT AAGGGAGATT TCGACCTTCT | 300 |
| GCCTAAGATA GTAGGTTTTA TGTTGGTACG GAGGGTCTGG GCAGTACAGA CACCATTCAC | 360 |
| GACTGTGGGC TTGGCGTTAA GAATTTCCGC GAATCTACGG CAAAGAGGGC TAATTTTCAT | 420 |
| Sense sequence | |
| SEQ ID NO: 32 | |
| ATGAAAATTA GCCCTCTTTG CCGTAGATTC GCGGAAATTC TTAACGCCAA GCCCACAGTC | 60 |
| GTGAATGGTG TCTGTACTGC CCAGACCCTC CGTACCAACA TAAAACCTAC TATCTTAGGC | 120 |
| AGAAGGTCGA AATCTCCCTT AACCATCCCA CAATTTCATT CTTTTGAAAG TAGTAAGAAG | 180 |
| AATGGTAGGG CTTTGTGTCT TGGTGAGACG GTCATTCTCA CTGAGGAGAT CAATCCGTAT | 240 |
| ATTTCAAGAC TTAGAGAACA TGGCATAAAG GTAACTGCCC TTCATAACCA CTGGCTATTT | 300 |
| ACCAACCCTA ATCTATGGTA TATCCACTAT GAAAAGATTG AGAAGCCTTT GGTCTTCGCT | 360 |
| AAAAGTGTAC GTGATGCCCT GAAAGTGTTA ACAAAAGAAA TCGTACGCCC GTGTAAATAG | 420 |
| Brevibacillus laterosporus PE36, GenBank: AXBT01000062.1 | |
| ACCESSION AXBT01000062 REGION: 312171 . . . 312590 | |
| /protein_id = “ERM16028.1” | |
| Antisense sequence | |
| SEQ ID NO: 33 | |
| CTATCTACAC GGGCGTACGA TTTCTTTTGT TAACACTTTC AGGGCATCAC GTACACTTTT | 60 |
| AGCGAAGACC AAAGGTTTCT CAATCTTTTC ATAGTGGATA TACCATAGAT TAGGGTTGGT | 120 |
| AAATAGCCAG TGGTTATGAA GGGCAGTTAC CTTTATGCCA TGTTCTCGAA GTCTTGAAAT | 180 |
| ATACGGATTG ATCTCCTCAG TGAGAATGAC CGTCTCACCA AGACACAAAG CCCTACCATT | 240 |
| CTTCTTAATG CTTTCAAAAG AATGAAATTG TGGGATGGTT AAGGGAGATT TCGACCTTCT | 300 |
| GCCTAAGATA GTAGGTTTTA TGTTGGTACG GAGGGTCTGG GCAGTACAGA CACCATTCAC | 360 |
| GACTGTGGGC TTGGCGTTAA GAATTTCCGC GAATCTACGG CAAAGAGGGC TAATTTTCAT | 420 |
| Sense sequence | |
| SEQ ID NO: 34 | |
| ATGAAAATTA GCCCTCTTTG CCGTAGATTC GCGGAAATTC TTAACGCCAA GCCCACAGTC | 60 |
| GTGAATGGTG TCTGTACTGC CCAGACCCTC CGTACCAACA TAAAACCTAC TATCTTAGGC | 120 |
| AGAAGGTCGA AATCTCCCTT AACCATCCCA CAATTTCATT CTTTTGAAAG CATTAAGAAG | 180 |
| AATGGTAGGG CTTTGTGTCT TGGTGAGACG GTCATTCTCA CTGAGGAGAT CAATCCGTAT | 240 |
| ATTTCAAGAC TTCGAGAACA TGGCATAAAG GTAACTGCCC TTCATAACCA CTGGCTATTT | 300 |
| ACCAACCCTA ATCTATGGTA TATCCACTAT GAAAAGATTG AGAAACCTTT GGTCTTCGCT | 360 |
| AAAAGTGTAC GTGATGCCCT GAAAGTGTTA ACAAAAGAAA TCGTACGCCC GTGTAGATAG | 420 |
| Brevibacillus laterosporus strain B9, GenBank: | |
| JNFS01000003.1 | |
| ACCESSION JNFS01000003 REGION: 927027 . . . 927446 | |
| Antisense sequence | |
| SEQ ID NO: 35 | |
| CTATTCACAC GGGCGTACGA TTTCTTTAGT TAACACTTTC AGGGCATCAC GTACACTTTT | 60 |
| AGCGAAGACC AAAGGTTCCT CAAGCTTTTC ATAGTGGATA TACCATAGAT TAGGGTCGGT | 120 |
| AAATAGCCAG TGGTTATGAA GGGCAGTTAC CTTTATGCCA TGTTCTCGAA GTCTTGAAAT | 180 |
| ATACGGATTG ATCTCCTCAG TGAGAATGAC TGTCTCACCA AGACACAAAG CCCTACCTTT | 240 |
| CTTCTTACTG TTTTCAAAAG AATGAAATTG TGGGATGGTT AAGGGAGATT TCGACCTTCT | 300 |
| GCCTAAGATA GTAGGTTTTA TGTTGGTACG AAGGGTCTGG GCAGTACAGA CACCATTAAC | 360 |
| AACTGTGGGC TCGGCGTTAA GAATTTTCGC GAATCTACGG CAAAGAGGGC TAATTTTCAT | 420 |
| Sense sequence | |
| SEQ ID NO: 36 | |
| ATGAAAATTA GCCCTCTTTG CCGTAGATTC GCGAAAATTC TTAACGCCGA GCCCACAGTT | 60 |
| GTTAATGGTG TCTGTACTGC CCAGACCCTT CGTACCAACA TAAAACCTAC TATCTTAGGC | 120 |
| AGAAGGTCGA AATCTCCCTT AACCATCCCA CAATTTCATT CTTTTGAAAA CAGTAAGAAG | 180 |
| AAAGGTAGGG CTTTGTGTCT TGGTGAGACA GTCATTCTCA CTGAGGAGAT CAATCCGTAT | 240 |
| ATTTCAAGAC TTCGAGAACA TGGCATAAAG GTAACTGCCC TTCATAACCA CTGGCTATTT | 300 |
| ACCGACCCTA ATCTATGGTA TATCCACTAT GAAAAGCTTG AGGAACCTTT GGTCTTCGCT | 360 |
| AAAAGTGTAC GTGATGCCCT GAAAGTGTTA ACTAAAGAAA TCGTACGCCC GTGTGAATAG | 420 |
| Brevibacillus laterosporus NCIMB 41419 (UNISS 18) | |
| Antisense sequence | |
| (SEQ ID NO: 37) | |
| CTATTTACAC GGGCGTACGA TTTCTTTTGT TAACACTTTC AGGGCATCAC GTACACTTTT | 60 |
| AGCGAAGACC AAAGGTTTCT CAATCTTTTC ATAGTGGATA TACCATAGAT TAGGGTTGGT | 120 |
| AAATAGCCAG TGGTTATGAA GGGCAGTTAC CTTTATGCCA TGTTCTCGAA GTCTTGAAAT | 180 |
| ATACGGATTG ATCTCATTAG TGAGAATGAC CGTCTCACCA AGACACAAAG CCCTACCATT | 240 |
| CTTCTTAATG CTTTCAAAAG AATGAAATTG TGGGATGGTT AAGGGAGATT TCGACCTTCT | 300 |
| GCCTAAGATA GTAGGTTTTA TGTTGGTACG GAGGGTCTGG GCAGTACAGA CACCATTCAC | 360 |
| GACTGTGGGC TTGGCGTTAA GAATTTCCGC GAATCTACGG CAAAGAGGGC TAATTTTCAT | 420 |
| UNISS18 | |
| Sense sequence | |
| SEQ ID NO: 38 | |
| ATGAAAATTA GCCCTCTTTG CCGTAGATTC GCGGAAATTC TTAACGCCAA GCCCACAGTC | 60 |
| GTGAATGGTG TCTGTACTGC CCAGACCCTC CGTACCAACA TAAAACCTAC TATCTTAGGC | 120 |
| AGAAGGTCGA AATCTCCCTT AACCATCCCA CAATTTCATT CTTTTGAAAG CATTAAGAAG | 180 |
| AATGGTAGGG CTTTGTGTCT TGGTGAGACG GTCATTCTCA CTAATGAGAT CAATCCGTAT | 240 |
| ATTTCAAGAC TTCGAGAACA TGGCATAAAG GTAACTGCCC TTCATAACCA CTGGCTATTT | 300 |
| ACCAACCCTA ATCTATGGTA TATCCACTAT GAAAAGATTG AGAAACCTTT GGTCTTCGCT | 360 |
| AAAAGTGTAC GTGATGCCCT GAAAGTGTTA ACAAAAGAAA TCGTACGCCC GTGTAAATAG | 420 |

FIG. 3 shows the alignment of the above-mentioned antisense sequences proving that the sequences have high identity among different Brevibacillus laterosporus strains. The alignment has been carried out by Basic Local Alignment-Search Tool (BLAST) of the National Center for Biotechnology Information (NCBI).

In addition, FIG. 4 shows the alignment of the polypeptide sequences codified by the above mentioned nucleic sequences.

### EXAMPLE 2: DESIGN OF PCR PRIMERS ON BREVIBACILLUS LATEROSPORUS SPECIES-SPECIFIC GENES WITH SEQUENCES SEQ ID NO:1 AND SEQ ID NO:2

In order to develop a system to detect Brevibacillus laterosporus, pairs of primers were designed to amplify regions of different size within the sequences SEQ ID NO:1 and SEQ ID NO:2

The sequences SEQ ID NO:1 and SEQ ID NO:2, used to design the primers, are shown below:

| Brevibacillus laterosporus LMG 15441 (ATCC 9141), GenBank: | |
|---|---|
| CP007806.1 | |
| ACCESSION CP007806 REGION: 373080 . . . 373850 | |
| SEQ ID NO: 1 | |
| TTATTTAAAT TGATCTGCTA CTAGTTGATC TAAGCTATTC TTTGCATCCT TTTCAGCCTT | 60 |
| AGCACCTGTA TCTTTGATCG AATCCTTCGC TTGTTTCTCC AATAATTTAG CTGATTCGTC | 120 |
| AACTACTTTT TGGATTTCAG CGACTTTTTC GTCGATAAAG CGTTTTGTTT CCTCTTTAGA | 180 |
| GCTAGCTTCT TTCTCACGAA TCAAACCATT AATTGTCTTT TTAGCATCGT TAATCGCACT | 240 |
| AGTTAGCTCT TGTTTTGCTT TGTCTTGCGC ACTGGTAACA TTCGTTTTTA CTTCTTGTCT | 300 |
| TCCTAGTTCT CCAGCTTGGT TCACTTTATT CGTAAGATCG ACTATCGAAT TGACTTTTGT | 360 |
| TTGATTGGCT GCTTTAGTAA TTTCCTGCTT TAATCCTTCT ACCTTCTGGT TGTATTGGTT | 420 |
| CTGCATATTG CCCTTGATTT CATTCGTTTT GCCTCTCAAA GCATTCAAAT AATCGTTTTT | 480 |
| AGCTGCCGTT ACTGCTGCAA CAGCACGATT TGCTTCTTCA CTACCTGCTT GCTTCACACG | 540 |
| ATCAGCAACC TGATTCTTTA TTTTATTAAG TTCAGCTTCA GTAGCTGCCT TTTTTTCCGT | 600 |
| TCCATACTTA AGCGCTTCAG CCGTAATAGA CGCCGATGAA GCAGCAAATT GTTTATCATA | 660 |
| CCAATTTTTT AGCTGCCCGC CTACATCAAC CGCCGCAAAT GCAGTACCTA AGCTACCAAT | 720 |
| CAGACCAACT GCCAACACAC TAATCACGAT TTTACTTTTT ACTTTTTTCA A | 771 |
| Brevibacillus laterosporus GI-9, GenBank: CAGD01000037.1 | |
| ACCESSION CAGD01000037 REGION: 7630 . . . 8049 | |
| SEQ ID NO: 2 | |
| CTATTTACAC GGGCGTACGA TTTCTTTTGT TAACACTTTC AGGGCATCAC GTACACTTTT | 60 |
| AGCGAAGACC AAAGGCTTCT CAATCTTTTC ATAGTGGATA TACCATAGAT TAGGGTTGGT | 120 |
| AAATAGCCAG TGGTTATGAA GGGCAGTTAC CTTTATGCCA TGTTCTCTAA GTCTTGAAAT | 180 |
| ATACGGATTG ATCTCCTCAG TGAGAATGAC CGTCTCACCA AGACACAAAG CCCTACCATT | 240 |
| CTTCTTACTA CTTTCAAAAG AATGAAATTG TGGGATGGTT AAGGGAGATT TCGACCTTCT | 300 |
| GCCTAAGATA GTAGGTTTTA TGTTGGTACG GAGGGTCTGG GCAGTACAGA CACCATTCAC | 360 |
| GACTGTGGGC TTGGCGTTAA GAATTTCCGC GAATCTACGG CAAAGAGGGC TAATTTTCAT | 420 |

The primers sequences used to amplify regions of the gene with sequence SEQ ID NO: 1 are shown below:

C28q F

(SEQ ID NO: 3)

5′-GCTTCACACGATCAGCAACC-3′
C28q R

(SEQ ID NO: 4)

5′-TGTAGGCGGGCAGCTAAAAA-3′;
PCR product size 155 bp
C28d1 F

(SEQ ID NO: 5)

5′-C AGC TTG GTT CAC TTT ATT CG-3′
C28d1 R

(SEQ ID NO: 6)

5′-TG AAG CAA GCA GGT AGT GAA-3′
PCR product size: 225 bp
C28d2 F

(SEQ ID NO: 7)

5′-CGT TTT TAC TTC TTG TCT TCC TAG-3′
C28d2 R

(SEQ ID NO: 8)

5′-AG GTT GCT GAT CGT GTG A-3′
PCR product size: 269 bp
C28d3 F

(SEQ ID NO: 9)

5′-AGC CTT AGC ACC TGT ATC TTT G-3′
C28d3 R

(SEQ ID NO: 10)

5′-AG GCA GCT ACT GAA GCT GA-3′
PCR product size: 536 bp
C28s F

(SEQ ID NO: 11)

5′-CTGCTACTAGTTGATCTAAG-3′
C28s R

(SEQ ID NO: 12)

5′-CTGATTGGTAGCTTAGGTA-3′;
PCR product size: 709 bp

The primers sequences used to amplify regions of the gene with sequence SEQ ID NO:2 are shown below:

C14d F

(SEQ ID NO: 13)

5′-CTA TTT ACA CGG GCG TAC G-3′
C14d R

(SEQ ID NO: 14)

5′-CAT TCT TTT GAA AGTAGTAAG AAG-3′;
Product size 264 bp
C14q 1F

(SEQ ID NO: 15)

5′-TCACCAAGACACAAAGCCCT-3′
C14q 1R

(SEQ ID NO: 16)

5′-CTCTTTGCCGTAGATTCGCG-3′;
Product size 193 bp
C14q 2F

(SEQ ID NO: 17)

5′-TCAGGGCATCACGTACACTT-3′
C14q 2R

(SEQ ID NO: 18)

5′-GGGCTTTGTGTCTTGGTGAG-3′
Product size 195 bp

In order to develop a detection system of Brevibacillus laterosporus, each primers pair was designed on the sequences SEQ ID NO:1 (ACCESSION CP007806 REGION: 373080 . . . 373850) and SEQ ID No:2 (ACCESSION CAGD01000037 REGION: 7630 . . . 8049) genes. Later these primers were tested in PCR reactions to amplify DNA extracted from a Brevibacillus laterosporus UNISS 18 overnight culture. PCR reactions were set in a total volume of 25 μl containing: 1× reaction buffer; 1.5 mM of MgCl2+; 100 ng DNA; 0.4 μM of each primer; 0.2 μM of each dNTPs; and 0.75 U of Taq DNA Polymerase. The reaction conditions were as follows: initial denaturation at 95° C. for 5 min, followed by 35 cycles of 95° C. for 30 s, 57° C. for 45 s and 72° C. for 45 s, followed by a final extension at 72° C. for 10 min. In FIG. 5 the PCR results show the expected band for each primers pair and since unspecific amplifications were not found the primers sequences designed for SEQ ID NO:1 and SEQ ID NO:2 can be used to detect Brevibacillus laterosporus.

### EXAMPLE 3: VALIDATION OF C28S AND C14D PRIMERS EFFICIENCY BY PCR AMPLIFICATION OF THE BREVIBACILLUS LATEROSPORUS DNA EXTRACTED WITH COMMERCIAL KIT

In order to test the efficiency of C28s and C14d primers pairs by PCR amplification, genomic DNA was extracted using commercial kit from five different Brevibacillus laterosporus strains: Brevibacillus laterosporus ATCC9141 (A1), Brevibacillus laterosporus ATCC6456 (A5), Brevibacillus laterosporus ATCC55122 (BOD), Brevibacillus laterosporus NI (new isolate), Brevibacillus laterosporus NCIMB 41419 (UNISS18). C28s and C14d primers pairs were used to detect genes with the sequences A and B (for instance SEQ ID NO:1 and SEQ ID NO:2 of Brevibacillus laterosporus LMG 15441 and of Brevibacillus laterosporus GI-9, respectively, or the corresponding sequences of other Brevibacillus laterosporus strains) encoding for a protein of SC-CSPB complex with a molecular weight around 28 kDa and for a protein of SC-CSPB complex with a molecular weight around 14 kDa, in two different PCR reactions. For each strain the two PCR reactions were set in a total volume of 25 μl containing: 1× reaction buffer; 1.5 mM of MgCl2+; 100 ng DNA; 0.4 μM of each primer; 0.2 μM of each dNTPs; and 0.75 U of Taq DNA Polymerase. The reaction conditions were as follows: initial denaturation at 95° C. for 5 min, followed by 35 cycles of 95° C. for 30 s, 57° C. for 45 s and 72° C. for 45 s, followed by a final extension at 72° C. for 10 min. FIG. 6 shows the PCR results on agarose gels: the expected band for the region of 709 bp of the sequence A encoding for a protein of SC-CSPB complex with a molecular weight around 28 kDa (for instance SEQ ID NO:1 of Brevibacillus laterosporus LMG 15441 or the corresponding sequences of other Brevibacillus laterosporus strains) was detected in all Brevibacillus laterosporus strains, whereas band for the region of 264 bp of the sequence B encoding for a protein of SC-CSPB complex with a molecular weight around 14 kDa (for instance SEQ ID NO:2 of Brevibacillus laterosporus GI-9 or the corresponding sequences of other Brevibacillus laterosporus strains) was found in Brevibacillus laterosporus NI and Brevibacillus laterosporus UNISSI8. Since unspecific amplification was not found, the results demonstrate the efficiency of both C28s and C14d primers in detecting genes with the above mentioned sequences.

### EXAMPLE 4: VALIDATION OF C28S AND C14D PRIMERS EFFICIENCY BY MULTIPLEX PCR AMPLIFICATION OF THE BREVIBACILLUS LATEROSPORUS DNA EXTRACTED WITH COMMERCIAL KIT

The efficiency of C28s and C14d primers pairs was also tested by multiplex PCR reactions. Therefore genomic DNA was extracted using commercial kit from five different Brevibacillus laterosporus strains: Brevibacillus laterosporus ATCC9141 (A1), Brevibacillus laterosporus ATCC6456 (A5), Brevibacillus laterosporus ATCC55122 (BOD), Brevibacillus laterosporus NI (new isolate), Brevibacillus laterosporus NCIMB 41419 (UNISS18). Multiplex PCR reactions were set up for the simultaneous detection of both genes with the sequences A and B (for instance SEQ ID NO:1 and SEQ ID NO:2 of Brevibacillus laterosporus LMG 15441 and of Brevibacillus laterosporus GI-9, respectively, or the corresponding sequences of other Brevibacillus laterosporus strains) encoding for a protein of SC-CSPB complex with a molecular weight around 28 kDa and for a protein of SC-CSPB complex with a molecular weight around 14 kDa, using C28s and C14d primers pairs. For each strain the PCR reaction was set in a total volume of 25 μl containing: 1× reaction buffer; 1.5 mM of MgCl2+; 100 ng DNA; 0.4 μM of each primer; 0.2 μM of each dNTPs; and 0.75 U of Taq DNA Polymerase. The reaction conditions were as follows: initial denaturation at 95° C. for 5 min, followed by 35 cycles of 95° C. for 30 s, 57° C. for 45 s and 72° C. for 45 s, followed by a final extension at 72° C. for 10 min. FIG. 7 shows multiplex PCR results on agarose gel: expected bands for genes with the sequences A and B (for instance SEQ ID NO:1 and SEQ ID NO:2 of Brevibacillus laterosporus LMG 15441 and of Brevibacillus laterosporus GI-9, respectively, or the corresponding sequences of other Brevibacillus laterosporus strains) encoding for a protein of SC-CSPB complex with a molecular weight around 28 kDa and for a protein of SC-CSPB complex with a molecular weight around 14 kDa were both detected in Brevibacillus laterosporus NI and Brevibacillus laterosporus UNISS18. In the other strains only the band relative to the gene with the sequence A encoding for a protein of SC-CSPB complex with a molecular weight around 28 kDa was detected. The absence of unspecific bands confirms the efficiency of C28s and C14d primers pairs.

### EXAMPLE 5: VALIDATION OF C28S AND C14D PRIMERS EFFICIENCY BY PCR AMPLIFICATION OF THE BREVIBACILLUS LATEROSPORUS DNA EXTRACTED WITH BOILING METHOD

To confirm the efficiency of C28s and C14d primers pairs by PCR amplification, genomic DNA from 5 different Brevibacillus laterosporus strains (Brevibacillus laterosporus ATCC9141 (A1), Brevibacillus laterosporus ATCC6456 (A5), Brevibacillus laterosporus ATCC55122 (BOD), Brevibacillus laterosporus NI (new isolate), Brevibacillus laterosporus NCIMB41419 (UNISS18) was extracted with boiling method. The DNA extraction was set up as follows: 1 mL of each overnight culture was boiled at 100° C. for 10 min and centrifuged at 12000 rpm for 5 min. The supernatant obtained was used as PCR template and C28s and C14d primers pairs were used to detect genes with sequences A and B (for instance SEQ ID NO:1 and SEQ ID NO:2 of Brevibacillus laterosporus LMG 15441 and of Brevibacillus laterosporus GI-9, respectively, or the corresponding sequences of other Brevibacillus laterosporus strains) encoding for a protein of SC-CSPB complex with a molecular weight around 28 kDa and for a protein of SC-CSPB complex with a molecular weight around 14 kDa, respectively, in two different PCR reactions. Therefore for each strain the two PCR reactions were set in a total volume of 25 μl containing: 1× reaction buffer; 1.5 mM of MgCl2+; 2 μl of DNA templete; 0.4 μM of each primer; 0.2 μM of each dNTPs; and 0.75 U of Taq DNA Polymerase. The reaction conditions were as follows: initial denaturation at 95° C. for 5 min, followed by 35 cycles of 95° C. for 30 s, 57° C. for 45 s and 72° C. for 45 s, followed by a final extension at 72° C. for 10 min. FIG. 8 shows the PCR results on agarose gels: expected band of 709 bp for gene with sequence A encoding for a protein of SC-CSPB complex with a molecular weight around 28 kDa (for instance SEQ ID NO:1 of Brevibacillus laterosporus LMG 15441 or the corresponding sequences of other Brevibacillus laterosporus strains) was detected in all Brevibacillus laterosporus strains, whereas the band of 264 bp for gene with sequence B encoding for a protein of SC-CSPB complex with a molecular weight around 14 kDa (for instance SEQ ID NO:2 of Brevibacillus laterosporus GI-9 or the corresponding sequences of other Brevibacillus laterosporus strains) was found in Brevibacillus laterosporus NI and Brevibacillus laterosporus UNISS18. Since unspecific amplification was not found, the results demonstrate the efficiency of both C28s and C14d primers in detecting genes with the above mentioned sequences.

### EXAMPLE 6: VALIDATION OF C28S AND C14D PRIMERS EFFICIENCY BY MULTIPLEX PCR AMPLIFICATION OF THE BREVIBACILLUS LATEROSPORUS DNA EXTRACTED WITH BOILING METHOD

The efficiency of C28s and C14d primers pairs was also tested by multiplex PCR reactions, using DNA extracted with boiling method. Therefore genomic DNA from 5 different Brevibacillus laterosporus strains (Brevibacillus laterosporus ATCC9141 (A1), Brevibacillus laterosporus ATCC6456 (A5), Brevibacillus laterosporus ATCC55122 (BOD), Brevibacillus laterosporus NI (new isolate), Brevibacillus laterosporus NCIMB 41419 (UNISS18) was extracted with boiling method as follows: 1 mL of each overnight culture was boiled at 100° C. for 10 min and centrifuged at 12000 rpm for 5 min. The supernatant obtained was used as PCR template. Therefore multiplex PCR reactions were set up for the simultaneous detection of both genes with sequences A and B (for instance SEQ ID NO:1 and SEQ ID NO:2 of Brevibacillus laterosporus LMG 15441 and of Brevibacillus laterosporus GI-9, respectively, or the corresponding sequences of other Brevibacillus laterosporus strains) encoding for a protein of SC-CSPB complex with a molecular weight around 28 kDa and for a protein of SC-CSPB complex with a molecular weight around 14 kDa, using C28s and C14d primers pairs. For each strain the PCR reaction was set in a total volume of 25 μl containing: 1× reaction buffer; 1.5 mM of MgCl2+; 2 μl of DNA templete; 0.4 μM of each primer; 0.2 μM of each dNTPs; and 0.75 U of Taq DNA Polymerase. The reaction conditions were as follows: initial denaturation at 95° C. for 5 min, followed by 35 cycles of 95° C. for 30 s, 57° C. for 45 s and 72° C. for 45 s, followed by a final extension at 72° C. for 10 min. FIG. 9 shows multiplex PCR results on agarose gels: expected bands corresponding to genes with sequences A and B encoding for a protein of SC-CSPB complex with a molecular weight around 28 kDa and for a protein of SC-CSPB complex with a molecular weight around 14 kDa were both detected in Brevibacillus laterosporus NI and Brevibacillus laterosporus UNISS18. In the other strains only the band corresponding to the gene with sequence A encoding for a protein of SC-CSPB complex with a molecular weight around 28 kDa was detected. The absence of unspecific bands confirms the efficiency of C28s and C14d primers pairs.

### EXAMPLE 7: VALIDATION OF THE SPECIES-SPECIFICITY OF C28S AND C14D PRIMERS BY PCR AMPLIFICATION OF BACTERIAL DNA EXTRACTED WITH COMMERCIAL KIT

In order to validate the species-specificity of C28s and C14d primers pairs by PCR amplification, genomic DNA was extracted, using commercial kit, from the following bacterial species: Photorhabdus luminescens, Paenibacillus xylanilyticus, Bacillus firmus, Bacillus psychrodurans, Bacillus megaterium, Bacillus amyloliquefaciens, Bacillus acquimaris, Paenibacillus lautus, Bacillus subtilis, Bacillus thuringiensis HD73, Bacillus thuringiensis HD567, Bacillus thuringiensis SA-11, Bacillus thuringiensis HD1, Brevibacillus laterosporus (new isolate), Brevibacillus laterosporus NCIMB41419 (UNISS 18). C28s and C14d primers pairs were used to detect genes with sequences SEQ ID NO:1 and SEQ ID NO:2 respectively, in two different PCR reactions. For each species the two PCR reactions were set in a total volume of 25 μl containing: 1×reaction buffer; 1.5 mM of MgCl2+; 100 ng DNA; 0.4 μM of each primer; 0.2 μM of each dNTPs; and 0.75 U of Taq DNA Polymerase. The reaction conditions were as follows: initial denaturation at 95° C. for 5 min, followed by 35 cycles of 95° C. for 30 s, 57° C. for 45 s and 72° C. for 45 s, followed by a final extension at 72° C. for 10 min. In FIG. 10 the PCR results show that the bands of the expected size corresponding to genes with sequences A and B encoding for a protein of SC-CSPB complex with a molecular weight around 28 kDa and for a protein of SC-CSPB complex with a molecular weight around 14 kDa were obtained only for Brevibacillus laterosporus strains. Therefore the species-specificity of these primers for Brevibacillus laterosporus DNA detection was demonstrated.

### EXAMPLE 8: VALIDATION OF THE SPECIES-SPECIFICITY OF C28S AND C14D PRIMERS BY MULTIPLEX PCR AMPLIFICATION OF DIFFERENT BACTERIAL DNA EXTRACTED WITH COMMERCIAL KIT

The species-specificity of C28s e C14d primers pairs was also tested by multiplex PCR reactions. Therefore genomic DNA was extracted, using commercial kit, from the following bacterial species: Photorhabdus luminescens, Paenibacillus xylanilyticus, Bacillus firmus, Bacillus psychrodurans, Bacillus megaterium, Bacillus amyloliquefaciens, Bacillus acquimaris, Paenibacillus lautus, Bacillus subtilis, Bacillus thuringiensis HD73, Bacillus thuringiensis HD567, Bacillus thuringiensis SA-11, Bacillus thuringiensis HD1, Brevibacillus laterosporus (new isolate), Brevibacillus laterosporus NCIMB41419 (UNISS 18). Multiplex PCR reactions were set up for the simultaneous detection of both genes with sequences A and B (for instance SEQ ID NO:1 and SEQ ID NO:2 of Brevibacillus laterosporus LMG 15441 and of Brevibacillus laterosporus GI-9, respectively, or the corresponding sequences of other Brevibacillus laterosporus strains) encoding for a protein of SC-CSPB complex with a molecular weight around 28 kDa and for a protein of SC-CSPB complex with a molecular weight around 14 kDa, using C28s and C14d primers pairs. For each microorganism the PCR reaction was set in a total volume of 25 μl containing: 1×reaction buffer; 1.5 mM of MgCl2+; 100 ng DNA; 0.4 μM of each primer; 0.2 μM of each dNTPs; and 0.75 U of Taq DNA Polymerase. The reaction conditions were as follows: initial denaturation at 95° C. for 5 min, followed by 35 cycles of 95° C. for 30 s, 57° C. for 45 s and 72° C. for 45 s, followed by a final extension at 72° C. for 10 min. In Figure lithe multiplex PCR results show the expected bands corresponding to genes with sequences A and B (for instance SEQ ID NO:1 and SEQ ID NO:2 of Brevibacillus laterosporus LMG 15441 and of Brevibacillus laterosporus GI-9, respectively, or the corresponding sequences of other Brevibacillus laterosporus strains) encoding for a protein of SC-CSPB complex with a molecular weight around 28 kDa and for a protein of SC-CSPB complex with a molecular weight around 14 kDa that were detected in Brevibacillus laterosporus strains. Therefore the species-specificity of these primers for Brevibacillus laterosporus DNA detection was confirmed.

### EXAMPLE 9: VALIDATION OF THE SPECIES-SPECIFICITY OF C28S AND C14D PRIMERS BY PCR AMPLIFICATION OF DIFFERENT BACTERIAL DNA EXTRACTED WITH BOILING METHOD

The species-specificity of C28s and C14d primers pairs was also tested by PCR amplification of genomic DNA extracted with boiling method from different bacterial species. Therefore the genomic DNA was extracted from the following bacterial species: Photorhabdus luminescens, Paenibacillus xylanilyticus, Bacillus firmus, Bacillus psychrodurans, Bacillus megaterium, Bacillus amyloliquefaciens, Bacillus acquimaris, Paenibacillus lautus, Bacillus subtilis, Bacillus thuringiensis HD73, Bacillus thuringiensis HD567, Bacillus thuringiensis SA-11, Bacillus thuringiensis HD1, Brevibacillus laterosporus (new isolate), Brevibacillus laterosporus NCIMB 41419 (UNISS 18). For each species the DNA extraction was set up as follows: 1 mL of each overnight culture was boiled at 100° C. for 10 min and centrifuged at 12000 rpm for 5 min. The supernatant obtained was used as PCR template and C28s and C14d primers pairs were used to detect genes with sequences A and B (for instance SEQ ID NO:1 and SEQ ID NO:2 of Brevibacillus laterosporus LMG 15441 and of Brevibacillus laterosporus GI-9, respectively, or the corresponding sequences of other Brevibacillus laterosporus strains) encoding for a protein of SC-CSPB complex with a molecular weight around 28 kDa and for a protein of SC-CSPB complex with a molecular weight around 14 kDa, respectively, in two different PCR reactions. Therefore for each species the two PCR reactions were set in a total volume of 25 μl containing: 1× reaction buffer; 1.5 mM of MgCl2+; 2 μl of DNA template; 0.4 μM of each primer; 0.2 μM of each dNTPs; and 0.75 U of Taq DNA Polymerase. The reaction conditions were as follows: initial denaturation at 95° C. for 5 min, followed by 35 cycles of 95° C. for 30 s, 57° C. for 45 s and 72° C. for 45 s, followed by a final extension at 72° C. for 10 min. In FIG. 12 the PCR results show the bands of the expected size corresponding to genes with sequences A and B encoding for a protein of SC-CSPB complex with a molecular weight around 28 kDa and for a protein of SC-CSPB complex with a molecular weight around 14 kDa only for Brevibacillus laterosporus strains. Therefore the species-specificity of these primers for Brevibacillus laterosporus DNA detection was confirmed.

### EXAMPLE 10: VALIDATION OF THE SPECIES-SPECIFICITY OF C28S AND C14D PRIMERS BY MULTIPLEX PCR AMPLIFICATION OF DIFFERENT BACTERIAL DNA EXTRACTED WITH BOILING METHOD

The species-specificity of C28s and C14d primers pairs was also tested by multiplex PCR amplification of genomic DNA extracted with boiling method from different bacterial species. Therefore the genomic DNA was extracted from the following bacterial species: Photorhabdus luminescens, Paenibacillus xylanilyticus, Bacillus firmus, Bacillus psychrodurans, Bacillus megaterium, Bacillus amyloliquefaciens, Bacillus acquimaris, Paenibacillus lautus, Bacillus subtilis, Bacillus thuringiensis HD73, Bacillus thuringiensis HD567, Bacillus thuringiensis SA-11, Bacillus thuringiensis HD1, Brevibacillus laterosporus (new isolate), Brevibacillus laterosporus NCIMB 41419 (UNISS 18). For each species the DNA extraction was set up as follows: 1 mL of each overnight culture was boiled at 100° C. for 10 min and centrifuged at 12000 rpm for 5 min. The supernatant obtained was used as PCR template. Therefore a multiplex PCR reactions were set up for the simultaneous detection of both genes with sequences A and B (for instance SEQ ID NO:1 and SEQ ID NO:2 of Brevibacillus laterosporus LMG 15441 and of Brevibacillus laterosporus GI-9, respectively, or the corresponding sequences of other Brevibacillus laterosporus strains) encoding for a protein of SC-CSPB complex with a molecular weight around 28 kDa and for a protein of SC-CSPB complex with a molecular weight around 14 kDa using C28s and C14d primers pairs. For each strain the PCR reaction was set in a total volume of 25 μl containing: 1× reaction buffer; 1.5 mM of MgCl2+; 2 μl of DNA templete; 0.4 μM of each primer; 0.2 μM of each dNTPs; and 0.75 U of Taq DNA Polymerase. The reaction conditions were as follows: initial denaturation at 95° C. for 5 min, followed by 35 cycles of 95° C. for 30 s, 57° C. for 45 s and 72° C. for 45 s, followed by a final extension at 72° C. for 10 min. In FIG. 13 the multiplex PCR results show the bands of the expected size corresponding to genes with sequences A and B (for instance SEQ ID NO:1 and SEQ ID NO:2 of Brevibacillus laterosporus LMG 15441 and of Brevibacillus laterosporus GI-9, respectively, or the corresponding sequences of other Brevibacillus laterosporus strains) encoding for a protein of SC-CSPB complex with a molecular weight around 28 kDa and for a protein of SC-CSPB complex with a molecular weight around 14 kDa in Brevibacillus laterosporus strains. Therefore the species-specificity of these primers for Brevibacillus laterosporus DNA detection was confirmed.

### EXAMPLE 11: VALIDATION OF C28S AND C14D PRIMERS FOR THE DETECTION OF BREVIBACILLUS LATEROSPORUS IN DIFFERENT MATRICES

In order to validate C28s and C14d primers pairs for the detection of Brevibacillus laterosporus, matrices of different nature were chosen and mixed with Brevibacillus laterosporus cultures. The experiments were set up using the following matrices: grapefruit juice, tomato puree, corn, milk, baby food, cat food, yogurt, sand and soil; the bacterial culture used were: Brevibacillus laterosporus ATCC9141 (A1), Brevibacillus laterosporus NC/MB 41419 (UNISS 18), Bacillus thuringiensis HD1 (negative control). For each experiment 1 mL of liquid matrix or 1 gr of solid matrix was mixed with each bacterial culture listed above and containing˜10*6 cell/mL. After mixing by vortex, the solution was used to extract genomic DNA with the boiling method as follows: 1 mL of each solution was boiled at 100° C. for 10 min and centrifuged at 12000 rpm for 5 min. The supernatant obtained was used as PCR template either in singleplex and multiplex PCR reaction with C28s and C14d pairs primers. Therefore the PCR reactions were set in a total volume of 25 μl containing: 1× reaction buffer; 1.5 mM of MgCl2+; 2 μl of DNA templete; 0.4 μM of each primer; 0.2 μM of each dNTPs; and 0.75 U of Taq DNA Polymerase. The reaction conditions were as follows: initial denaturation at 95° C. for 5 min, followed by 35 cycles of 95° C. for 30 s, 57° C. for 45 s and 72° C. for 45 s, followed by a final extension at 72° C. for 10 min. In FIG. 14 the PCR results show the detection of the expected band corresponding to the gene with sequence A encoding for a protein of SC-CSPB complex with a molecular weight around 28 kDa in Brevibacillus laterosporus ATCC9141 (A1) and Brevibacillus laterosporus UNISS18 samples.

FIG. 15 shows the detection genes with sequences A and B (for instance SEQ ID NO:1 and SEQ ID NO:2 of Brevibacillus laterosporus LMG 15441 and of Brevibacillus laterosporus GI-9, respectively, or the corresponding sequences of other Brevibacillus laterosporus strains) encoding for a protein of SC-CSPB complex with a molecular weight around 28 kDa and for a protein of SC-CSPB complex with a molecular weight around 14 kDa in multiplex reaction. These results confirm that, among matrices from different nature, C28s and C14d primers pairs are efficient in detecting Brevibacillus laterosporus.

### EXAMPLE 12: VALIDATION OF C28S PRIMERS FOR THE DETECTION OF BREVIBACILLUS LATEROSPORUS IN INSECT

The efficiency of C28s and C14d primers pairs was tested in detecting Brevibacillus laterosporus in insects either wild-caught or laboratory-raised. Samples of Apis mellifera and Rhynchophorus ferrugineus were caught from the wild, whereas samples of Musca domestica were raised in laboratory on a diet lacking Brevibacillus laterosporus (untreated control) or on a diet containing either Brevibacillus laterosporus ATCC9141 (A1) or Brevibacillus laterosporus NCIMB 41419 (UNISS18) or Bacillus thuringiensis israelensis (negative control). Each insect was ground in 1 mL/5 mL of sterile MIIQ H2O and the supernatant was recovered by centrifugation. Later the DNA was extracted with the boiling method as follows: the recovered supernatant was boiled at 100° C. for 10 min, centrifuged at 12000 rpm for 5 min and 5 μl were used as PCR template. The PCR reaction was set up using the C28s primers pair in a total volume of 25 μl containing: 1× reaction buffer; 1.5 mM of MgCl2+; 0.4 μM of each primer; 0.2 μM of each dNTPs; and 0.75 U of Taq DNA Polymerase. The reaction conditions were as follows: initial denaturation at 95° C. for 5 min, followed by 35 cycles of 95° C. for 30 s, 57° C. for 45 s and 72° C. for 45 s, followed by a final extension at 72° C. for 10 min. In FIG. 16 the PCR results show the band of 709 bp corresponding to the gene with sequence A (for instance SEQ ID NO:1 of Brevibacillus laterosporus LMG 15441 or the corresponding sequences of other Brevibacillus laterosporus strains) encoding for a protein of SC-CSPB complex with a molecular weight around 28 kDa in samples of Apis mellifera and Rhynchophorus ferrugineus. In fact it is known that Brevibacillus laterosporus is a common inhabitant of the body of certain insects like Apis mellifera. In the case of Musca domestica the band of 709 bp corresponding to the gene with sequence A encoding for a protein of SC-CSPB complex with a molecular weight around 28 kDa was obtained only for insect specimens fed with Brevibacillus laterosporus. The results confirm the efficiency of C28s primers pair in detecting Brevibacillus laterosporus in the insect body.

### EXAMPLE 13: VALIDATION OF C28Q PRIMERS EFFICIENCY BY QUANTITATIVE REAL-TIME PCR FOR THE DETECTION OF BREVIBACILLUS LATEROSPORUS IN INSECTS

C28q primers were designed to detect Brevibacillus laterosporus by quantitative real-time PCR (qPCR) on DNA extracted from Apis mellifera adults. Therefore the total honey bee DNA was extracted with commercial kit, quantified and used to prepare DNA standards for qPCR. The efficiency of C28q primers was validated by six 2-fold serial dilution standards, starting from 100 ng/μl down to 3.125 ng/μl of total honey bee DNA. For each standard two different reactions were set up using C28q primers and EF1 primers separately, in order to detect specifically the Brevibacillus laterosporus gene with sequence A (for instance SEQ ID NO:1 of Brevibacillus laterosporus LMG 15441 or the corresponding sequences of other Brevibacillus laterosporus strains) encoding for a protein of SC-CSPB complex with a molecular weight around 28 kDa and honey bee EF1 housekeeping gene. Then the qPCR reactions were performed in a total volume of 20 μl containing: 1×SYBR Green PCR Master Mix; 0.5 μM of each primer and 3 μl of DNA templete. For each standard point the Ct values were detected for the Brevibacillus laterosporus gene with sequence A encoding for a protein of SC-CSPB complex with a molecular weight around 28 kDa and Apis mellifera EF1 housekeeping gene as show in Table 1. These results demonstrate that C28q primers can be used in quantitative Real-Time PCR methods to detect Brevibacillus laterosporus DNA from a pool of total insect DNA.

TABLE 1

| Gene | | | | DNA starting | | DNA final | |
|---|---|---|---|---|---|---|---|
| Detected | Task | Quantity | Ct value | concentration | | concentration | |
| Brevibacillus | Negative | 0 | Undetermined | | | | |
| laterosporus | control | | | | | | |
| SEQ N. 1 | | | | | | | |
| Brevibacillus | Standard | 1 | 26.93717 | 100 | ng/μl | 15 | ng/μl |
| laterosporus | | | | | | | |
| SEQ N. 1 | | | | | | | |
| Brevibacillus | Standard | 0.5 | 27.585375 | 50 | ng/μl | 7.5 | ng/μl |
| laterosporus | | | | | | | |
| SEQ N. 1 | | | | | | | |
| Brevibacillus | Standard | 0.25 | 28.142847 | 25 | ng/μl | 3.75 | ng/μl |
| laterosporus | | | | | | | |
| SEQ N. 1 | | | | | | | |
| Brevibacillus | Standard | 0.125 | 29.046078 | 12.5 | ng/μl | 1.875 | ng/μl |
| laterosporus | | | | | | | |
| SEQ N. 1 | | | | | | | |
| Brevibacillus | Standard | 0.062 | 30.138021 | 6.25 | ng/μl | 0.937 | ng/μl |
| laterosporus | | | | | | | |
| SEQ N. 1 | | | | | | | |
| Brevibacillus | Standard | 0.031 | 30.383854 | 3.125 | ng/μl | 0.468 | ng/μl |
| laterosporus | | | | | | | |
| SEQ N. 1 | | | | | | | |
| Apis mellifera | Negative | 0 | Undetermined | | | | |
| E1F | control | | | | | | |
| Apis mellifera | Standard | 1 | 21.931072 | 100 | ng/μl | 15 | ng/μl |
| E1F | | | | | | | |
| Apis mellifera | Standard | 0.5 | 22.682041 | 50 | ng/μl | 7.5 | ng/μl |
| E1F | | | | | | | |
| Apis mellifera | Standard | 0.25 | 23.612026 | 25 | ng/μl | 3.75 | ng/μl |
| E1F | | | | | | | |
| Apis mellifera | Standard | 0.125 | 24.441343 | 12.5 | ng/μl | 1.875 | ng/μl |
| E1F | | | | | | | |
| Apis mellifera | Standard | 0.062 | 25.427956 | 6.25 | ng/μl | 0.937 | ng/μl |
| E1F | | | | | | | |
| Apis mellifera | Standard | 0.031 | 26.397243 | 3.125 | ng/μl | 0.468 | ng/μl |
| E1F | | | | | | | |
| Brevibacillus | Negative | 0 | Undetermined | | | | |
| laterosporus | control | | | | | | |
| SEQ N. 1 | | | | | | | |

### EXAMPLE 14: IDENTIFICATION OF SPORE SURFACE PROTEINS CORRESPONDING TO SEQ ID NO 19 ON BREVIBACILLUS LATEROSPORUS SPORES

Synchronized cultures of Brevibacillus laterosporus strain UNISS 18 were used for surface proteins extraction and separation from spores. Proteins from the spore coat-canoe shaped parasporal body complex (SC-CSPB) were extracted from pure spore suspensions harvested by centrifugation at 13,000 rpm for 10 min, and re-suspended in 1 ml 0.1N NaOH-1% thioglycollic acid. The suspension was titrated adding 1 M NaOH until pH 11.5 and centrifuged at 13,000 rpm for 10 min. The supernatant was dialysed at 4° C. against water using SnakeSkin™ Pleated Dialysis tubing, 3,500 MWCO (Cole-Parmer Instrument Company, UK). Water was changed three times after 1 h, 2 h and overnight, and then the supernatant was collected for analysis. Protein samples were mixed with Laemmli buffer, boiled for 5 min and run in a 10% or 15% SDS-PAGE gel using a Mini-Protrean electrophoresis system (BioRad Laboratories Inc., USA). Gels were stained with Coomassie and digitized with an ImageScanner III (GE Healthcare).

Individual gel regions corresponding to major protein bands from different polyacrylamide gels were manually excised, destained, reduced, carbamidomethylated, and trypsin digested. Tryptic peptides were submitted to LC MS/MS analysis using a XCT Ultra 6340 ion trap equipped with a 1200 HPLC system and a chip cube (Agilent Technologies, Palo Alto, Calif.). Mass spectrometry output data were analyzed on the software provided by the manufacturer (6300 Series Ion Trap LCMS) employing Mascot Daemon MS/MS ion search software (Version 2.3, Matrix Science, Boston, Mass.) for protein identification. Data were then processed against the NCBI database (http://www.ncbi.nlm.nih.gov).

As shown in FIG. 17, the 1-DE profile of surface proteins specifically extracted by alkali and a reducing agent showed a major band corresponding to a molecular weight around 28 kDa. As a result of LC-MS/MS analysis, protein corresponding to SEQ ID NO 19 was identified.

This experiment demonstrates the actual and abundant expression of protein corresponding to SEQ ID NO 19, which confirms the possibility to target this protein through methods known in the art (for example ELISA, Western Blot) for the detection of Brevibacillus laterosporus, and especially Brevibacillus laterosporus spores.

### EXAMPLE 15: DETERMINATION OF THE LEVEL OF EXPRESSION OF GENE WITH SEQUENCE A IN COMPARISON TO 16S RRNA GENE

The purpose of this experiment was to demonstrate the higher level of expression of gene corresponding to sequence A in respect to 16S rRNA gene in Brevibacillus laterosporus, in order to prove the achievement of a significantly higher efficiency of the method of the present invention targeting sequence A gene, in comparison to other detection methods based on 16S rRNA gene detection. The relative expression levels of sequence A gene and 16S rRNA gene mRNAs were examined by quantitative real-time PCR (qRT-PCR). For this purpose, total RNA was extracted from 24 h cultures of Brevibacillus laterosporus NCIMB41419 (UNISS18). The bacterial samples were immediately resuspended in TRIzol®Reagent (Life Technologies) before being subjected to sonication and cooling in ice. Then, extrated RNA (1 μg), treated with RQ1 RNase-Free DNase (Promega), was reverse transcribed to complementary DNA (cDNA) with SuperScript® II Reverse Transcriptase and RNaseOUT™ Recombinant Ribonuclease Inhibitor using a mix of oligo(dT) and random hexamer primers according to manufacturer's instructions (Life Technologies). Quantitative PCR experiments were carried out soon after the synthesis of cDNA. Reactions were conducted using Power SYBR® Green PCR Master Mix and were run on an Applied Biosystems 7900HT Fast Real-Time PCR System according to manufacturer's instructions (Life Technologies) and with following cycle conditions: 50° C. 2 min, 95° C. 10 min, 95° C. 15 s and 60° C. 1 min (40 cycles), 60° C. 1 min. Used primers, respectively forward and reverse, were 5′-GCTTCACACGATCAGCAACC-3′ (SEQ ID NO:3) and 5′-TGTAGGCGGGCAGCTAAAAA-3′ (SEQ ID NO:4) for cDNA corresponding to sequence A, and 5′-TGTAGCGGTGAAATGCGTAG-3′(SEQ ID NO:47) and 5′-GCGGCACTAAGGGTATTGAA-3′ (SEQ ID NO:48) designed on Brevibacillus laterosporus 16S rRNA gene (GeneBank Acc. NO. NR_112212) used as reference gene. The primers pairs efficiency was evaluated by standard curve and dissociation curve analyses, and according to the manufacturer's manual. For each primers pair the dissociation curve was set increasing gradually the temperature from 60° C. to 95° C. after the real-time PCR reaction. To exclude genomic DNA contaminations, reactions included controls lacking template or reverse transcriptase. Samples were run in three technical replicates. Three independent experiments with different batches of bacterial cultures were conducted. Real time qPCR data were analyzed using 1 Way-ANOVA followed by Least Significant Difference (LSD) tests for post-hoc comparison of means. As a result, sequence A gene showed a significantly higher level of expression in comparison to 16S rRNA (p<0.05). In terms of threshold cycle (Ct), means±SE were as follow: 17.52±0.44 for SEQ. A gene; 27.68±0.71 for 16S rRNA.

It can be concluded that, since Brevibacillus laterosporus produces a higher number of mRNA copy (=higher expression) of SEQUENCE A gene in comparison to 16S rRNA gene, a higher efficiency in detecting or quantifying Brevibacillus laterosporus by RT-PCR or RT-qPCR, respectively, is achievable with the method of the present invention based on SEQUENCE A, in respect to methods known in the art and based on 16S rRNA.

### EXAMPLE 16: DETERMINATION OF THE LEVEL OF EXPRESSION OF GENES WITH SEQUENCES A, DURING DIFFERENT BACTERIAL STAGES

The purpose of this experiment was to determine the level of expression of gene corresponding to sequence A in Brevibacillus laterosporus during different bacterial stage of growth. For this purpose aliquots of synchronized bacterial cultures were harvested at consecutive time intervals (12, 24, 36 h), corresponding to exponential, stationary and sporulation phases, respectively, as confirmed by phase microscopy observations. The relative expression levels of sequence A gene and of the reference gene 16S rRNA were then examined by quantitative real-time PCR (qRT-PCR). For this purpose, total RNA was extracted from harvested cultures of Brevibacillus laterosporus NCIMB41419 (UNISS18). The bacterial samples were immediately resuspended in TRIzoI®Reagent (Life Technologies) before being subjected to sonication and cooling in ice. Then, extrated RNA (1 μg), treated with RQ1 RNase-Free DNase (Promega), was reverse transcribed to complementary DNA (cDNA) with SuperScript® II Reverse Transcriptase and RNaseOUT™ Recombinant Ribonuclease Inhibitor using a mix of oligo(dT) and random hexamer primers according to manufacturer's instructions (Life Technologies). Quantitative PCR experiments were carried out soon after the synthesis of cDNA. Reactions were conducted using Power SYBR® Green PCR Master Mix and were run on an Applied Biosystems 7900HT Fast Real-Time PCR System according to manufacturer's instructions (Life Technologies) and with following cycle conditions: 50° C. 2 min, 95° C. 10 min, 95° C. 15 s and 60° C. 1 min (40 cycles), 60° C. 1 min. Used primers, respectively forward and reverse, were 5′-GCTTCACACGATCAGCAACC-3′ (SEQ ID NO:3) and 5′-TGTAGGCGGGCAGCTAAAAA-3′ (SEQ ID NO:4) for cDNA corresponding to sequence A, and 5′-TGTAGCGGTGAAATGCGTAG-3′ (SEQ ID NO: 47) and 5′-GCGGCACTAAGGGTATTGAA-3′ (SEQ ID NO:48) designed on Brevibacillus laterosporus 16S rRNA gene (GeneBank Acc. NO. NR_112212) used as reference gene. The primers pairs efficiency was evaluated by standard curve and dissociation curve analyses, and according to the manufacturer's manual. For each primers pair the dissociation curve was set increasing gradually the temperature from 60° C. to 95° C. after the real-time PCR reaction. To exclude genomic DNA contaminations, reactions included controls lacking template or reverse transcriptase. Samples were run in three technical replicates. Three independent experiments with different batches of bacterial cultures were conducted. Real time qPCR data were analyzed using 1 Way-ANOVA followed by Least Significant Difference (LSD) tests for post-hoc comparison of means. As a result, SEQUENCE A gene showed a significant increase in the level of expression during the sporulation phase (p<0.05), as shown by FIG. 18. It can be concluded that, the expression level of SEQUENCE A is always significantly higher than 16S rRNA and increases over bacterial growth toward sporulation, which is in relation to the increased synthesis of the encoded protein (SEQ ID NO 19) that is part of the spore coat-canoe shaped parasporal body complex. This means that the method of detection using primers designed on SEQUENCE A allows the detection of Brevibacillus laterosporus in any stage of growth and may also allow to monitor the state of bacterial growth.

### EXAMPLE 17: COMPARATIVE EXPERIMENT OF BREVIBACILLUS LATEROSPORUS DETECTION USING SEQUENCE A AND 16S RRNA BASED METHODS

The purpose of this experiment was to show the higher efficiency of targeting Sequence A gene for the detection of Brevibacillus laterosporus in a matrix, in respect to targeting 16S rRNA. For this purpose, PCR amplification was based on cDNA obtained by RNA in vitro transcription. Total RNA was extracted from homogenized pools of 10 Apis mellifera workers employing TRIzol® Reagent (Life Technologies) according to manufacturer's protocol. All RNA samples were treated with RQ1 RNase-Free DNase (Promega) and an aliquot (1 μg) of each was used to synthesize first-strand cDNA employing oligo dT (Promega), SuperScript® II Reverse Transcriptase (Life Technologies) and RNaseOUT™ Recombinant Ribonuclease Inhibitor (Life Technologies) according to the manufacturers' instructions. Serial dilutions of the template (1:10; 1:100; 1:1000) were prepared by diluting cDNA samples with sterile water. The PCR reactions were set up in a total volume of 25 μl containing: 1× reaction buffer; 1.5 mM of MgCl2+; 0.4 μM of each primer; 0.2 μM of each dNTPs; and 0.75 U of Taq DNA Polymerase. The reaction conditions were as follows: initial denaturation at 95° C. for 5 min, followed by 30 cycles of 95° C. for 40 s, 61° C. for 40 s and 72° C. for 40 s, followed by a final extension at 72° C. for 10 min. An aliquot (1 μl) o the previously mentioned serial dilutions was used in different PCR reactions. The following primers pairs were used in different PCR reactions using the same templates: primers pair SEQ ID NO:11 and SEQ ID NO:12 targeting Sequence A; primers pair SEQ ID NO:3 and SEQ ID NO:4 targeting Sequence A; primer set L25 5′-TGAAGCGAAACGGAAAG-3′ (SEQ ID NO: 49) and R322 5′-CGTCAAGGTGCTACCTTATT-3′ (SEQ ID NO: 50) targeting 16s rRNA (CN101956018A); primer pairs 5′-TGTAGCGGTGAAATGCGTAG-3′ (SEQ ID NO:47) and 5′-GCGGCACTAAGGGTATTGAA-3′ (SEQ ID NO:48) which have been designed on Brevibacillus laterosporus 16S rRNA gene (GeneBank Acc. NO. NR_112212); primer pairs BREV174F 5′-AGACCGGGATAACATAGGGAAACTTAT-3′ (SEQ ID No:51) and 1377R 5′-GGCATGCTGATCCGCGATTACTAGC-3′ (SEQ ID NO:52) targeting 16s rRNA (Shida et al., 1996).

Table 2 shows the higher detection efficiency when targeting SEQUENCE A instead of 16S rRNA, which is in relation to the higher expression level of Sequence A gene. Namely, table 2 shows the detection of Brevibacillus laterosporus from an insect based matrix (Apis mellifera workers) by reverse transcription polymerase chain reaction (RT-PCR) using different primer pairs targeting Sequence A or 16S rRNA and different dilutions of cDNA template.

### Table 2

TABLE 2

| | | | DETECTION RESULT | | |
|---|---|---|---|---|---|
| | | | (+) = positive | | |
| | | | (−) = negative | | |
| | TARGET | | Serial dilutions | | |
| PRIMER PAIRS | GENE | Reference | 1:10 | 1:100 | 1:1000 |
| 5′-CTGCTACTAGTTGATCTAAG-3′ | Sequence | SEQ ID NO: 11 | + | + | + |
| and | A | and | | | |
| 5′-CTGATTGGTAGCTTAGGTA-3′ | | SEQ ID NO: 12 | | | |
| 5′-GCTTCACACGATCAGCAACC-3′ | Sequence | SEQ ID NO: 3 | + | + | + |
| and | A | and | | | |
| 5′-TGTAGGCGGGCAGCTAAAAA-3′ | | SEQ ID NO: 4 | | | |
| 5′-TGAAGCGAAACGGAAAG-3′ | 16S rRNA | SEQ ID 49 (L25) and | + | − | − |
| and | | SEQ IDNO: 50 | | | |
| 5′-CGTCAAGGTGCTACCTTATT-3′ | | (R322) | | | |
| | | Patent (China) | | | |
| | | CN101956018A | | | |
| 5′-TGTAGCGGTGAAATGCGTAG-3′ | 16S rRNA | SEQ ID NO: 47 and 48 | + | − | − |
| and | | Designed on sequence | | | |
| 5′-GCGGCACTAAGGGTATTGAA-3′ | | with GeneBank Acc. | | | |
| | | NO. NR_112212 | | | |
| 5′-AGACCGGGATAACATAGGGAAA | 16S rRNA | SEQ ID NO: 51 | + | − | − |
| CTTAT-3′ | | (BREV174F) | | | |
| and | | and | | | |
| 5′-GGCATGCTGATCCGCGATTACTA | | SEQ ID NO: 52 (1377R) | | | |
| GC-3′ | | Shida et al., 1996 | | | |

The results of this experiment confirm the higher efficiency of the method of the present invention targeting Sequence A in detecting Brevibacillus laterosporus, in comparison with other detection methods based on 16S rRNA. In other terms, the method of the present invention allows to detect/quantify Brevibacillus laterosporus contained in a matrix at significantly lower concentrations.

### REFERENCES

Djukic M., Poehlein A., Thürmer A., Daniel R., 2011. Genome sequence of Brevibacillus laterosporus LMG 15441, a pathogen of invertebrates. Journal of Bacteriology 193: 5535-5536.
Fits-James, P. C., Young, I. E., 1958. Morphological and chemical studies of the spores and parasporal bodies of Bacillus laterosporus. J. Biophys. Biochem. Cytol. 4,639-649.
Li, G., Xu, J., Song, W., Ye, W., Dong, G., Zhu, L., Guo, L. Full genome sequence of Brevibacillus laterosporus strain B9; a biological control strain isolated from Zhejiang, China [Unpublished]. Submitted to gene bank (28 May 2014) State Key Lab for Rice Biology, China National Rice Research Institute, Tiyuchang Road 359, Hangzhou, Zhejiang 310006, China.
Ruiu L., Floris I., Satta A., Ellar D. J., 2007. Toxicity of a Brevibacillus laterosporus strain lacking parasporal crystals against Musca domestica and Aedes aegypti. Biological Control, vol. 43; p. 136-143.
Ruiu L., 2013. Brevibacillus laterosporus, a Pathogen of Invertebrates and a Broad-Spectrum Antimicrobial Species. Insects, vol. 4; p. 476-492.
Shida, O., Takagi, H., Kadowaki, K., Komagata K., 1996. Proposal for two new genera, Brevibacillus gen. nov. and Aneurinobacillus gen. nov. International Journal of Systematic Bacteriology 46: 939-946.
Sharma, V., Singh, P. K., Midha, S., Ranjan, M., Korpole, S., Patil, P. B., 2012. Genome sequence of Brevibacillus laterosporus strain GI-9 Journal of Bacteriology
Theodore, C. M., Stamps, B. W., King, J. B., Price, L. S. L., Powell, D. R., Stevenson, B. S., Cichewicz, R. H., 2014. Genomic and metabolomic insights into the natural product biosynthetic diversity of a feral-hog-associated Brevibacillus laterosporus strain. PLoS ONE 9 (3), e90124

1) Marker for use in the detection and/or quantification of Brevibacillus laterosporus in a sample, said marker consisting of a nucleic acid sequence encoding a surface polypeptide of the spore coat and the canoe shaped parasporal body of Brevibacillus laterosporus, said nucleic acid sequence comprising or consisting of:
a) SEQ ID NO: 1;
b) SEQ ID NO: 2;
c) a fragment of the nucleic acid sequences a) or b), said fragment having at least 12 bp, preferably from 15 to 30 bp, more preferably from 18 to 24 bp; or
d) a nucleic acid sequence having a sequence identity of at least 80%, preferably 90% with any of the nucleic acid sequences a)-c);
e) a complement nucleic sequence of said sequences a)-d).

2) Marker for use in the detection and/or quantification of Brevibacillus laterosporus in a sample, said marker consisting of a surface polypeptide sequence of the spore coat and the canoe shaped parasporal body of Brevibacillus laterosporus, said surface polypeptide sequence comprising or consisting of:
f) SEQ ID NO: 19;
g) SEQ ID NO: 20;
h) a fragment of the polypeptide sequence f) or g) having at least 5 aminoacids, preferably from 6 to 20 aminoacids, more preferably from 8 to 15 aminoacids;
i) a polypeptide sequence having a sequence identity of at least 90% with any of the polypeptide sequences f)-h).

3) Marker according to claim 1, wherein Brevibacillus laterosporus is chosen from the group consisting of Brevibacillus laterosporus ATCC9141, Brevibacillus laterosporus ATCC6456, Brevibacillus laterosporus BOD ATCC 55122, Brevibacillus laterosporus NCIMB 41419, Brevibacillus laterosporus GI-9, Brevibacillus laterosporus PE36, Brevibacillus laterosporus 89, Brevibacillus laterosporus DSM25 (ATCC 64).

4) Method for the detection and/or quantification of Brevibacillus laterosporus in a sample, said method comprising or consisting of the detection and/or quantification of at least one marker selected from the group consisting of:
a nucleic acid sequence comprising or consisting of SEQ ID NO:1 or the complement nucleic acid sequence thereof; a fragment of said SEQ ID NO:1 or complement nucleic acid sequence thereof, said fragment having at least 12 bp, preferably from 15 to 30 bp, more preferably from 18 to 24 bp; a nucleic acid sequence having a sequence identity of at least 80%, preferably 90% with said SEQ ID NO: 1, complement nucleic acid sequence or said fragment;
a surface polypeptide sequence comprising or consisting of SEQ ID NO: 19, a fragment thereof having at least 5 aminoacids, preferably from 6 to 20 aminoacids, more preferably from 8 to 15 aminoacids;
a polypeptide sequence having a sequence identity of at least 90% with said SEQ ID NO: 19 or fragment thereof.

5) Method according to claim 4, wherein Brevibacillus laterosporus is chosen from the group consisting of Brevibacillus laterosporus ATCC9141, Brevibacillus laterosporus ATCC6456, Brevibacillus laterosporus BOD ATCC 55122, Brevibacillus laterosporus NCIMB 41419, Brevibacillus laterosporus GI-9, Brevibacillus laterosporus PE36, Brevibacillus laterosporus 89, Brevibacillus laterosporus DSM25 (ATCC 64).

6) Method for the detection and/or quantification in a sample of Brevibacillus laterosporus, said method comprising or consisting of the detection of at least one marker selected from the group consisting of:
a nucleic acid sequence comprising or consisting of: SEQ ID NO:2 or the complement nucleic acid sequence thereof; a fragment of said SEQ ID NO:2 or complement nucleic acid sequence thereof, said fragment having at least 12 bp, preferably from 15 to 30 bp, more preferably from 18 to 24 bp; or
a nucleic acid sequence having a sequence identity of at least 80%, preferably 90% with said SEQ ID NO: 2, the complement nucleic acid sequence or said fragment;
a surface polypeptide sequence comprising or consisting of: SEQ ID NO: 20, a fragment thereof having at least 5 aminoacids, preferably from 6 to 20 aminoacids, more preferably from 8 to 15 aminoacids;
a polypeptide sequence having a sequence identity of at least 90% with said SEQ ID NO: 20 or fragment thereof.

7) Method according to claim 6, wherein Brevibacillus laterosporus is Brevibacillus laterosporus NCIMB 41419.

8) Method according to claim 4, said method being carry out by means of PCR and/or Real Time PCR techniques by the use of at least one of the following primer pairs:
(SEQ ID NO: 3)
5′-GCTTCACACGATCAGCAACC-3′
(SEQ ID NO: 4)
5′-TGTAGGCGGGCAGCTAAAAA-3′;
(SEQ ID NO: 5)
5′-C AGC TTG GTT CAC TTT ATT CG-3′
(SEQ ID NO: 6)
5′-TG AAG CAA GCA GGT AGT GAA-3′;
(SEQ ID NO: 7)
5′-CGT TTT TAC TTC TTG TCT TCC TAG-3′
(SEQ ID NO: 8)
5′-AG GTT GCT GAT CGT GTG A-3′;
(SEQ ID NO: 9)
5′-AGC CTT AGC ACC TGT ATC TTT G-3′
(SEQ ID NO: 10)
5′-AG GCA GCT ACT GAA GCT GA-3′;
(SEQ ID NO: 11)
5′-CTGCTACTAGTTGATCTAAG-3′
(SEQ ID NO: 12)
5′-CTGATTGGTAGCTTAGGTA-3′;
preferably
(SEQ ID NO: 11)
5′-CTGCTACTAGTTGATCTAAG-3′
(SEQ ID NO: 12)
5′-CTGATTGGTAGCTTAGGTA-3′
or
(SEQ ID NO: 3)
5′-GCTTCACACGATCAGCAACC-3′
(SEQ ID NO: 4)
5′-TGTAGGCGGGCAGCTAAAAA-3′.

9) Method according to claim 6, said method being carry out by means of PCR and/or real Time PCR techniques by the use of at least one of the following primer pairs:
(SEQ ID NO: 13)
5′-CTA TTT ACA CGG GCG TAC G-3′
(SEQ ID NO: 14)
5′-CAT TCT TTT GAA AGTAGTAAG AAG-3′;
(SEQ ID NO: 15)
5′-TCACCAAGACACAAAGCCCT-3′
(SEQ ID NO: 16)
5′-CTCTTTGCCGTAGATTCGCG-3′;
(SEQ ID NO: 17)
5′-TCAGGGCATCACGTACACTT-3′
(SEQ ID NO: 18)
5′-GGGCTTTGTGTCTTGGTGAG-3′;
preferably
(SEQ ID NO: 13)
5′-CTA TTT ACA CGG GCG TAC G-3′
(SEQ ID NO: 14)
5′-CAT TCT TTT GAA AGTAGTAAG AAG-3′

10) Method according to claim 4, further comprising or consisting of the detection of at least one marker selected from the group consisting of:
a nucleic acid sequence comprising or consisting of: SEQ ID NO:2 or the complement nucleic acid sequence thereof; a fragment of said SEQ ID NO:2 or complement nucleic acid sequence thereof, said fragment having at least 12 bp, preferably from 15 to 30 bp, more preferably from 18 to 24 bp; or
a nucleic acid sequence having a sequence identity of at least 80%, preferably 90% with said SEQ ID NO: 2, the complement nucleic acid sequence or said fragment;
a surface polypeptide sequence comprising or consisting of: SEQ ID NO: 20, a fragment thereof having at least 5 aminoacids, preferably from 6 to 20 aminoacids, more preferably from 8 to 15 aminoacids;
a polypeptide sequence having a sequence identity of at least 90% with said SEQ ID NO: 20 or fragment thereof.

11) Kit for the detection and/or quantification of Brevibacillus laterosporus in a sample, said kit comprising or consisting of at least one of the following primer pairs:
(SEQ ID NO: 3)
5′-GCTTCACACGATCAGCAACC-3′
(SEQ ID NO: 4)
5′-TGTAGGCGGGCAGCTAAAAA-3′;
(SEQ ID NO: 5)
5′-C AGC TTG GTT CAC TTT ATT CG-3′
(SEQ ID NO: 6)
5′-TG AAG CAA GCA GGT AGT GAA-3′;
(SEQ ID NO: 7)
5′-CGT TTT TAC TTC TTG TCT TCC TAG-3′
(SEQ ID NO: 8)
5′-AG GTT GCT GAT CGT GTG A-3′;
(SEQ ID NO: 9)
5′-AGC CTT AGC ACC TGT ATC TTT G-3′
(SEQ ID NO: 10)
5′-AG GCA GCT ACT GAA GCT GA-3′;
(SEQ ID NO: 11)
5′-CTGCTACTAGTTGATCTAAG-3′
(SEQ ID NO: 12)
5′-CTGATTGGTAGCTTAGGTA-3′;
preferably
(SEQ ID NO: 11)
5′-CTGCTACTAGTTGATCTAAG-3′
(SEQ ID NO: 12)
5′-CTGATTGGTAGCTTAGGTA-3′-
or
(SEQ ID NO: 3)
5′-GCTTCACACGATCAGCAACC-3′
(SEQ ID NO: 4)
5′-TGTAGGCGGGCAGCTAAAAA-3′.
together with suitable reactive agents for the detection and/or quantification.

12) Kit according to claim 11, wherein Brevibacillus laterosporus is chosen from the group consisting of Brevibacillus laterosporus ATCC9141, Brevibacillus laterosporus ATCC6456, Brevibacillus laterosporus BOD ATCC 55122, Brevibacillus laterosporus NCIMB 41419, Brevibacillus laterosporus GI-9, Brevibacillus laterosporus PE36, Brevibacillus laterosporus 89, Brevibacillus laterosporus DSM25 (ATCC 64).

13) Kit for the detection and/or quantification in a sample of Brevibacillus laterosporus, said kit comprising or consisting of at least one of the following primer pairs:
(SEQ ID NO: 13)
5′-CTA TTT ACA CGG GCG TAC G-3′
(SEQ ID NO: 14)
5′-CAT TCT TTT GAA AGTAGTAAG AAG-3′;
(SEQ ID NO: 15)
5′-TCACCAAGACACAAAGCCCT-3′
(SEQ ID NO: 16)
5′-CTCTTTGCCGTAGATTCGCG-3′;
(SEQ ID NO: 17)
5′-TCAGGGCATCACGTACACTT-3′
(SEQ ID NO: 18)
5′-GGGCTTTGTGTCTTGGTGAG-3′;
preferably
(SEQ ID NO: 13)
5′-CTA TTT ACA CGG GCG TAC G-3′
(SEQ ID NO: 14)
5′-CAT TCT TTT GAA AGTAGTAAG AAG-3′
together with suitable reactive agents for the detection and/or quantification.

14) Kit according to claim 13, wherein Brevibacillus laterosporus is Brevibacillus laterosporus NCIMB 41419.

15) Kit for the detection and/or quantification in a sample of Brevibacillus laterosporus, said kit comprising or consisting of at least one of the following primer pairs:
(SEQ ID NO: 3)
5′-GCTTCACACGATCAGCAACC-3′
(SEQ ID NO: 4)
5′-TGTAGGCGGGCAGCTAAAAA-3′;
(SEQ ID NO: 5)
5′-C AGC TTG GTT CAC TTT ATT CG-3′
(SEQ ID NO: 6)
5′-TG AAG CAA GCA GGT AGT GAA-3′;
(SEQ ID NO: 7)
5′-CGT TTT TAC TTC TTG TCT TCC TAG-3′
(SEQ ID NO: 8)
5′-AG GTT GCT GAT CGT GTG A-3′;
(SEQ ID NO: 9)
5′-AGC CTT AGC ACC TGT ATC TTT G-3′
(SEQ ID NO: 10)
5′-AG GCA GCT ACT GAA GCT GA-3′;
(SEQ ID NO: 11)
5′-CTGCTACTAGTTGATCTAAG-3′
(SEQ ID NO: 12)
5′-CTGATTGGTAGCTTAGGTA-3′;
preferably
(SEQ ID NO: 11)
5′-CTGCTACTAGTTGATCTAAG-3′
(SEQ ID NO: 12)
5′-CTGATTGGTAGCTTAGGTA-3′-
or
(SEQ ID NO: 3)
5′-GCTTCACACGATCAGCAACC-3′
(SEQ ID NO: 4)
5′-TGTAGGCGGGCAGCTAAAAA-3′.
together with suitable reactive agents for the detection and/or quantification; and at least one of the following primer pairs:
(SEQ ID NO: 13)
5′-CTA TTT ACA CGG GCG TAC G-3′
(SEQ ID NO: 14)
5′-CAT TCT TTT GAA AGTAGTAAG AAG-3′;
(SEQ ID NO: 15)
5′-TCACCAAGACACAAAGCCCT-3′
(SEQ ID NO: 16)
5′-CTCTTTGCCGTAGATTCGCG-3′;
(SEQ ID NO: 17)
5′-TCAGGGCATCACGTACACTT-3′
(SEQ ID NO: 18)
5′-GGGCTTTGTGTCTTGGTGAG-3′;
preferably
(SEQ ID NO: 13)
5′-CTA TTT ACA CGG GCG TAC G-3′
(SEQ ID NO: 14)
5′-CAT TCT TTT GAA AGTAGTAAG AAG-3′
together with suitable reactive agents for the detection and/or quantification.
